(19) United States
(12) Patent Application Publication
(10) Pub. No.: US 2019/0035497 A1
(43) Pub. Date: Jan. 31, 2019

(54) METHOD AND APPARATUS FOR LABELING AND MANAGING INVENTORY OF MEDICINAL SUBSTANCES

(71) Applicant: CODONICS, INC., Middleburg Heights, OH (US)

(72) Inventors: Michael KOLBERG, Hinkley, OH (US); Michael GRABEL, Middleburg Heights, OH (US); Gary KEEFE, Brecksville, OH (US); Ross GOODMAN, Solon, OH (US); Stephen MUCHER, Yellow Springs, OH (US); Lawrence SRNKA, Northfield Center, OH (US)

(21) Appl. No.: 16/085,660

(22) PCT Filed: Mar. 16, 2017
(86) PCT No.: PCT/US17/22734

§ 371 Date: Sep. 17, 2018

Related U.S. Application Data
(60) Provisional application No. 62/408,797, filed on Oct. 16, 2016.
(60) Provisional application No. 62/308,984, filed on Mar. 16, 2016.

(51) Int. Cl. G16H 20/10; G06K 7/14; G06K 7/10

(52) U.S. Cl. CPC G16H 20/10; G06K 2007/10524; G06K 7/10881; G06K 7/1413

(57) ABSTRACT

Provided is a remote barcode reader that is to communicate with a terminal in a medical network. The remote barcode reader includes an optical barcode scanner that transmits a signal indicative of a barcode in response to interrogating the barcode. A non-transitory computer-readable memory stores, at least temporarily, information obtained in response to reading the barcode. A network interface communicates wirelessly over a wireless communication channel with a remote device in a medical network to obtain information pertaining to a drug that is identifiable from the information obtained in response to reading the barcode. A processor initiates the transmission of a communication based on the information obtained from the barcode to the remote device, and delays transmitting at least a portion of the information obtained from the barcode until at least a time when a response including information related to the drug is received from the remote device.

### BACKGROUND OF THE INVENTION

### 1. Field of the Invention

This application relates generally to a method and apparatus for labeling and managing inventory of medicinal substances and, more specifically, to monitoring usage of medicinal substances at individual stocking locations to determine inventory levels and, more specifically, to aggregating information about the usage of medicinal substances at remote locations to manage inventory, maintain patient medical records, perform billing related functions and track the disposal or waste of drugs.

### 2. Description of Related Art

It is common for medicinal substances, such as drugs for example, to be stocked at multiple locations in a healthcare facility. Healthcare facilities, such as hospitals for example, frequently maintain many types of drugs at substantial inventory levels in operating rooms to support a variety of different surgical procedures.

Locations such as operating rooms are sometimes referred to as ORs. These are often at remote locations in the hospital relative to areas where drugs are stored such as the pharmacy for example. This can create a challenge for hospital personnel in monitoring, replenishing and maintaining the inventory of drugs in these remote locations.

A common technique to manage drug inventories in ORs is to establish the minimum amount of an item, such as a drug vial or ampoule for example, that must be in stock to meet demand for a given period, such as a day or a week. This minimum stock level for an item is sometimes called a PAR level. Items with inventory levels that are below the established PAR level are restocked so the total number of the item at that location equals or exceeds the PAR level. A maximum stocking level can also be established that is greater than the PAR level.

Drugs stocked in the OR are commonly stored in bins or designated areas within a locked cart sometimes called an “anesthesia cart”. Some anesthesia carts are basic with pull out drawers, manual locks and limited automation. These are sometimes called “dumb carts”.

Another type of cart, sometimes called a “smart cart”, can have significant automation built-in to the cart with integrated computers, barcode scanners and other technologies that perform additional functions including electronically controlling the dispensing of some drugs. The electronically dispensed drugs are typically those drugs designated as controlled substances. Controlled substances are drugs or substances whose manufacture, possession, or use is regulated by a government (e.g., designated as Schedule II Controlled Substances, Schedule III Controlled Substances, as established in U.S. Drug Enforcement Administration regulations, 21 C.F.R. Sections 1308.11 through 1308.15). It should be noted that most drugs stored in anesthesia carts are not designated as controlled substances, and the dispensing of these drugs by smart carts is not controlled as strictly as the dispensing of drugs that are controlled substances.

Maintaining inventory levels in dumb carts is usually done manually by counting the drugs in each bin or designated storage area within the cart drawers and restocking the drugs by adding the number of units necessary to meet or exceed the established PAR levels for those drugs. This process can be time consuming for healthcare personnel especially when repeated in a large number of remote locations such as ORs.

Maintaining inventory levels in smart carts usually relies on the automation features of the cart. For drugs that are designated controlled substances, the drug vials or ampoules are commonly dispensed by the cart individually per a clinician request using the automation features of the cart. This enables the cart to maintain an accurate count of each drug dispensed. Other drugs stored in the cart that are not controlled substances are typically not dispensed individually, but can be retrieved as needed. Clinicians commonly open the cart drawers and remove such drugs from their bin or storage areas in much that same way that drugs are removed from dumb carts. However, the clinician is expected to use the automation features of the cart to record the removal of the drug by manually entering the drug information into the cart computer or scanning the barcode on the label of the drug vial or ampoule that identifies the drug using a barcode scanner that is part of the automation system of the cart to update the inventory of the drug.

Because operating rooms are often high stress environments where clinicians are frequently challenged to deliver patient care, omissions in recording drugs removed from the anesthesia cart, such as a failure to scan drugs with the barcode scanner connected to smart carts, can occur and result in inaccurate inventory reporting on the cart automation system. Manual inventory checks are therefore needed to ensure accurate stock levels are maintained. This negates some of the benefits of smart carts.

Once drugs are removed from either a dumb cart or a smart cart, the drugs are typically prepared one-at-a-time by transferring the drug from each vial or ampoule into another container, such as a syringe for example, that is labeled with information about the drug in its final form in the syringe.

The process of creating the label for the syringe commonly requires scanning the NDC code encoded in the barcode on the manufacturer's label of the vial or ampoule on a drug labeling device that is separate from, and operates independently of the anesthesia cart to create the label. The NDC is a code used in the U.S. to identify the drug in a container. The drug labeling device identifies the drug from the NDC code and prints a secondary label with the required information that is to be affixed to the syringe to properly identify the drug contained in the syringe.

Although the anesthesia cart and the drug labeling device operate independently of each other, the drug labeling device can be physically attached to (e.g., rests on top of), or is positioned in close proximity to, the anesthesia cart. The proximity of the drug labeling device to the anesthesia cart and the clinical function it performs of printing the syringe labels required by federal regulation promotes high compliance by clinicians in scanning drugs removed from anesthesia carts.

The high level of compliance by clinicians in scanning the barcode of the drug vial or ampoule on the drug labeling device to generate the secondary label is driven by regulatory requirements for printing a label with proper drug identification when the drug is transferred to a dispensing container such as a syringe, for example. This is in contrast to the lower compliance of scanning the vial on the automation system of the smart cart for inventory management because that activity can sometimes be a distraction from the task of delivering patient care in the OR, and may be deemed unnecessary by some clinicians because of the less stringent monitoring of drugs that are not controlled substances.

### BRIEF SUMMARY OF THE INVENTION

Accordingly, there is a need in the art for a method and apparatus for recording drug inventory usage to maintain proper inventory levels at remote locations in healthcare facilities without interfering the with normal workflow of clinicians engaged in the delivery of health care.

According to one aspect, the subject application involves a remote barcode reader that is to communicate with a terminal in a medical network. The remote barcode reader includes an optical barcode scanner that transmits a signal indicative of a barcode in response to interrogating the barcode. A non-transitory computer-readable memory stores, at least temporarily, information obtained in response to reading the barcode. A network interface communicates wirelessly over a wireless communication channel with a remote device in a medical network to obtain information pertaining to a drug that is identifiable from the information obtained in response to reading the barcode. A processor initiates the transmission of a communication based on the information obtained from the barcode to the remote device, and delays transmitting at least a portion of the information obtained from the barcode until at least a time when a response including information related to the drug is received from the remote device.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

The invention may take physical form in certain parts and arrangement of parts, embodiments of which will be described in detail in this specification and illustrated in the accompanying drawings which form a part hereof and wherein:

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows an illustrative embodiment of a computer terminal for drug labeling;

FIG. 2 schematically shows an illustrative embodiment of components included as part of the embodiment of the computer terminal shown in FIG. 1;

FIG. 3 shows an illustrative arrangement of a communication network at a healthcare facility;

FIG. 4 shows an illustrative embodiment of a drug cart and drug cart controller provided with a barcode scanner;

FIG. 5 shows an illustrative embodiment of a drug cart and drug cart controller provided with a barcode scanner and a multiplexer that establishes communications between a drug labeling terminal and a drug cart controller;

FIG. 6 shows another illustrative embodiment of a drug cart and drug cart controller provided with a barcode scanner in communication with a drug labeling terminal;

FIG. 7 shows another illustrative embodiment of a drug cart and drug cart controller provided with a barcode scanner and a drug labeling terminal in communication with the drug cart controller;

FIG. 8A shows a schematic illustration of an embodiment of a multiplexer;

FIG. 8B shows another schematic illustration of an embodiment of a multiplexer;

FIG. 9 shows an illustrative embodiment of a drug cart and drug cart controller provided in communication with a drug labeling terminal via wireless interfaces over a wireless communication channel;

FIG. 10 shows an illustrative embodiment of a drug cart and drug cart controller, an electronic health record system in communication with a gas anesthesia machine, and a drug labeling terminal in communication with each other via wireless interfaces over a wireless communication channel;

FIG. 11 shows an illustrative embodiment of a mobile stand supporting a remote barcode reader in communication with a monitor added to the network shown in FIG. 10;

FIG. 12 is a block diagram showing a schematic view of a remote barcode reader according to an embodiment of the present disclosure; and

FIG. 13 shows an illustrative embodiment of a mobile stand supporting a remote barcode reader in hardwired-communication with an electronic health record system provided adjacent to a gas anesthesia system.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

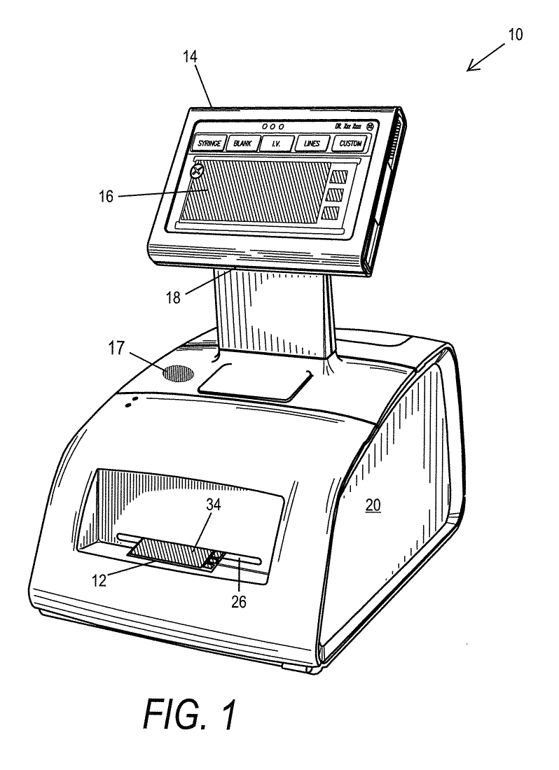

As shown in FIG. 1, the computer terminal 10 includes a touch-screen display 14 that can be pivotally coupled to a cabinet 20 to display a virtual label 16 comprising label content 34 that will be printed onto a label 12 that will be applied to a medicinal substance. The computer terminal 10 can be operable to scan a computer-readable code and print a label to be applied to a medical container such as a syringe as described in U.S. Pat. No. 9,262,585 to Keefe et al., which is incorporated by reference herein in its entirety. The display 14 can display soft keys that, when touched by a technician or any other user, inputs data and commands into the computer terminal 10. The virtual label 16 is a computer-generated rendering of the label 12 that offers the user visual confirmation of the appearance of the physical label 12 to be printed by a printer 26. A computer-input peripheral such as a non-contact scanner 18 can be provided at a convenient location, such as integrally formed in a bottom portion of the display 14 to read a machine-readable code supported beneath the scanner 18 for example. Integrally forming the scanner 18 as part of the display 14 provides for space savings over an arrangement where the scanner 18 is formed as a separate peripheral, which can be repositioned relative to the display 14. However, other embodiments can allow for a separate and distinct scanner 18 and/or display 14.

The computer-input peripheral can be a barcode reader or radio-frequency identification (“RFID”) tag reader, or any other device that reads a machine-readable code such as a barcode or RFID code, respectively, or any other machine-readable code without requiring contact between the computer terminal and the code, and optionally the user during entry of the code. According to alternate embodiments, the display 14 can be utilized by a user as the computer-input peripheral. For such embodiments, the soft keys displayed by the display 14 can be selected to input information such as a medicinal substance being prepared to be administered to a patient or other information to be utilized in generating the label as described herein. According to yet alternate embodiments, a speaker 17 can optionally be provided to the display 14 or any other portion of the computer terminal 10 to broadcast audible sounds.

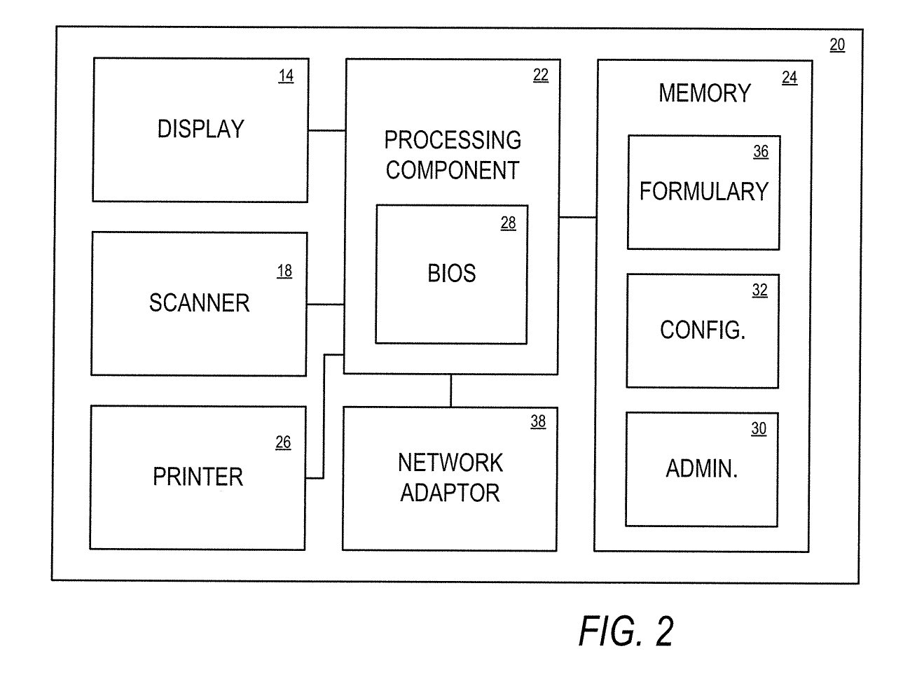

The computer terminal 10 also includes a cabinet 20 that houses or supports components that are operable to produce the label 12 in compliance with a medical labeling standard. But if what is being labeled is anything other than the medicinal substance, then the label 12 produced is to be compliant with a standard developed by a trade or professional organization, governing body, government agency, a healthcare provider or facility such as a hospital, or any other standards body setting forth policies for labeling such material. The internal components housed within the cabinet 20 are schematically illustrated by the block diagram of FIG. 2. The components can be formed from an arrangement of computer hardware such as ASICs, computer processors, programmable logic controllers and other circuitry; or a combination of computer hardware and computer-executable instructions. For example, a processing component 22 is provided to execute computer-executable instructions stored in a non-transitory, computer-readable memory 24 such as a hard disk drive, read-only memory (“ROM”), random access memory (“RAM”), optical disc, or any other suitable memory device, or any combination thereof. The computer-executed instructions, when executed by the computer processor 22, result in the performance of the method of generating a label for a medicinal substance described in detail below. A BIOS 28 is provided to load the operating system and other such administrative instructions 30 stored in the memory 24 and manage hardware interface permissions of the computer terminal 10. The operating system can be configured to only load authorized updates to prevent unauthorized changes to the formulary 36, configuration data 32 and administration instructions 30. Configuration data 32 controls various features of the computer terminal 10 that are active and available for use at any given time. The configuration data 32 can optionally be stored, updated and deleted from the memory 24 by the introduction of a so-called smart drive comprising a USB compatible flash memory to the computer terminal 10. When the smart drive is introduced to the computer terminal 10, it establishes the configuration data 32 of the computer terminal 10. The configuration data 32 can optionally be used to deactivate functional features that the computer terminal 10 would otherwise be able to perform based on the model of the computer terminal 10 purchased. Accordingly, a common hardware platform of the computer terminal 10 can be configured in a plurality of different functional configurations based on the configuration data 32.

In addition to the administrative instructions 30, the memory 24 also stores an updatable formulary 36 containing a database of medicinal substances that can be identified by the computer terminal 10 and select information for each medicinal-substance entry in the database. The formulary 36 can optionally be stored, updated and deleted from the memory 24 by the introduction of a so-called smart drive comprising a USB compatible flash memory to the computer terminal 10. When the smart drive is introduced to the computer terminal 10, it establishes the formulary 36 of the computer terminal 10. Illustrative examples of the select information that can be provided for the medicinal-substance entries includes, but is not limited to, an ID number such as a National Drug Code (“NDC”), UPC code, EAN code, or any other identifying data that can be used to relate a barcode or other computer-readable code to the medicinal-substance entries; a sound file that, when played, audibly announces the name of the medicinal substance identified in response to scanning a machine readable code; warning data; or any combination thereof.

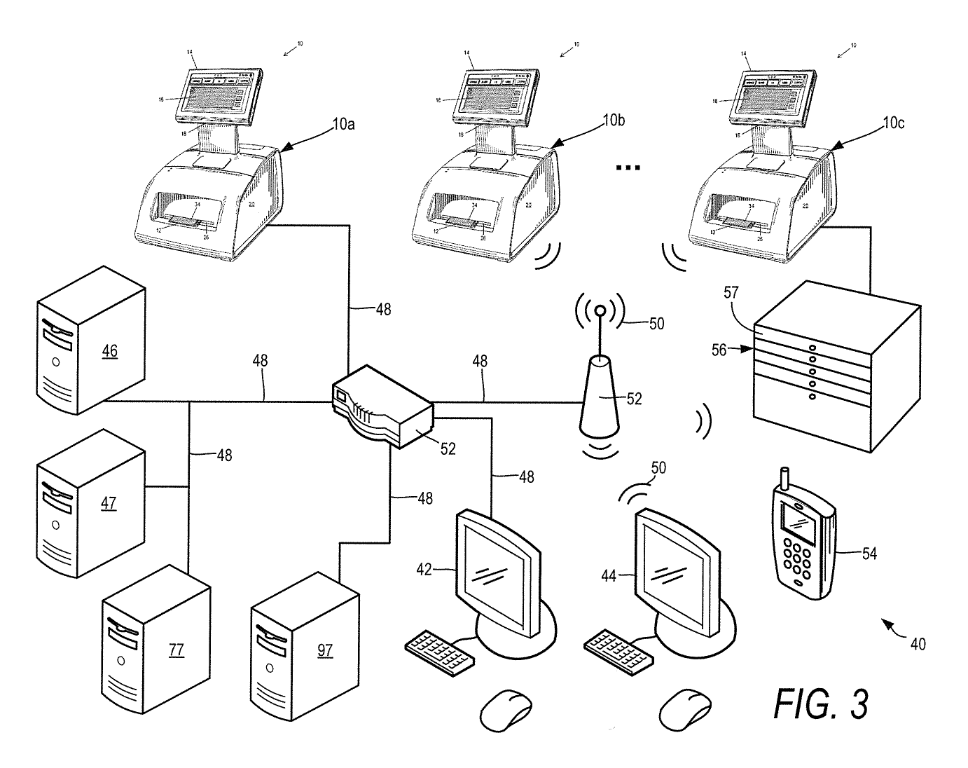

Embodiments of the formulary 36 can also optionally include quantity data associated with one, a plurality or each of the drugs in the formulary 36. The drugs having a field indicative of the number of single use vials, for example, remaining in a certain drug cart 56 associated with the computer terminal 10c, as shown in FIG. 3, can optionally be monitored by the computer terminal 10 and/or a remote terminal such as a pharmacy terminal 42 described below, for example, to ensure a sufficient supply of those drugs is available from the cart 56. According to one embodiment, drugs to be monitored can be associated with a minimum threshold field that indicates the minimum quantity of single use vials, for example, that must be stored by the cart 56 as a minimum inventory, as established on a case-by-case basis by the health-care facility where the cart 56 is located. Similarly, another field is associated with a number indicating the actual number of single use vials of at least a portion, and optionally each drug present in the cart.

A network adaptor 38 is operatively connected to communicate with the processing component 22 for translating signals received by the computer terminal 10 over a network 40 at a medical facility, such as that illustrated in FIG. 3. The network adaptor 38 can be compatible with any type of network communication. For example, the network adaptor 38 can include a hardwired, 10Base-T, 100Base-T, or 1000Base-T Ethernet interface with an RJ-45 socket, a coaxial cable interface, a fiber-optic interface, any format of wireless communication interface such as an antenna compatible with any of the 802.11 standards established by the IEEE, or any combination thereof. Embodiments including wireless network adaptors 38 can employ any desired securing protocol such as WEP, WPA and WPA2, for example, and other suitable security protocol. For embodiments including a network adaptor 38 compatible to communicate over a plurality of different network communication channels, both a hard-wired communication portion of the network adaptor 38 and a wireless communication portion of the network adaptor 38 can optionally be concurrently active. Thus, the computer terminal 10 can optionally communicate via both the hard-wired and wireless portions of the network adaptor 38 concurrently.

As shown in FIG. 3, a plurality of the computer terminals, each referred to generally at 10 and separately at 10a, 10b, 10c, can be included in a network 40 at a healthcare facility. For example, each operating room in which surgical procedures take place may have one of the computer terminals 10 located therein. Other networks may include a computer terminal 10 in an examination room where procedures such as minimally invasive examinations of patients are conducted. Each of the computer terminals 10a, 10b, 10c can be provided with a unique identifier, which can be stored electronically in the memory 24 (e.g., as part of the configuration data 32 or administration data 30, or separately from such data, etc.), to uniquely identify those terminals 10a, 10b, 10c. The identifier of each terminal 10a, 10b, 10c can also optionally be stored in association with the respective location of those terminals, allowing each of the terminals 10a, 10b, 10c to be allocated to a particular OR

The network 40 also includes a pharmacy computer terminal 42 executing computer-executable instructions (referred to hereinafter as an administration tool or “AT”) that, when executed, manage one or more, and optionally all of the computer terminals 10. Each computer terminal 10 to be managed by the AT can be optionally assigned a user-specified designation using the AT to distinguish the computer terminals from each other on the network 40, and to optionally provide the user with a brief description of each computer terminal 10. For example, a computer terminal 10 located in operating room #1 can be assigned the designation OR-1 to indicate its location. According to alternate embodiments, the user-specified name Cart-1 could be assigned to a computer terminal on mobile cart #1. An IT computer terminal 44 can also optionally be connected as part of the network 40 to execute the AT and allow technical personnel to manage technical aspects of the computer terminals 10, but optionally exclude from the permissions granted to technical personnel the ability to alter drug or other medical-related content stored by the computer terminals 10. The permissions granted to a user at the terminals 42, 44 can optionally be determined when the user logs in based on a username/password combination, a computer-readable identification, or any other identifying information. Thus, the terminals 42, 44 do not necessarily have to be dedicated solely for any particular purpose.

The pharmacy terminal 42 can be located in a pharmacy at a healthcare facility, where an inventory of controlled drugs and medicinal substances (hereinafter generally referred to as “drugs”) is maintained. A pharmacist or a plurality of pharmacists maintain and administer a master drug database (“MDD”) containing an identity, identification code (e.g., NDC) number, concentration and other pertinent information for drugs used by the pharmacy. Drugs are entered into the MDD by the pharmacist, and the terminals 42, 44, and optionally other terminals connected to the network 40 can restrict access to the MDD and prevent unauthorized individuals from entering or altering drug entries in the MDD, and optionally from accessing the MDD altogether. In other words, the pharmacist(s) registered and authorized to work at the health care facility and those they grant permission to access the MDD are the only individuals permitted to manipulate data in the MDD.

From the MDD, the pharmacist manages a formulary to be stored in the memory 24 of one or more of the computer terminals 10 using the AT with the pharmacist permission. The formulary can include a subset, but less than the entirety of the MDD, and the subset can optionally comprise drugs that are commonly used in the operating room or other locations at the healthcare facility where the computer terminal 10 is positioned. The same formulary can optionally be stored in the memory 24 of more than one computer terminal, and can optionally be customized to include drugs utilized during surgical procedures relating to a particular medical discipline. For example, the same formulary comprising drugs commonly used during cardiac surgical procedures may be stored in the memory 24 of computer terminals 10a, 10b, which are each located in a respective operating room dedicated for such procedures. Another, different formulary comprising drugs, optionally in appropriate doses, suitable to be administered to children can be stored in the memory 24 of a computer terminal 10 located in an operating room dedicated for pediatric surgical procedures. According to alternate embodiments, the formulary 36 stored in the memory 24 of a computer terminal 10 can be evaluated and updated, replaced or otherwise changed before each surgical procedure if the operating room where the computer terminal 10 is located is not dedicated for a particular type of surgical procedure. By way of example, a subset, but fewer than all of the drugs in the MDD designated as suitable for use with children can be made selectable using the computer terminal 10 during a pediatric surgical procedure. When a formulary update is needed to accommodate a specific type of procedure, a pharmacist's access can be required to update, replace or otherwise change the formulary in the computer terminals 10, and updating, replacing and changing the formulary in the memory 24 in each of the computer terminals 10 can be performed over the network as described in detail below.

In addition to a pharmacist's level of permission, there can be other permission levels limiting access to the computer terminals 10 to different users. For example, an anesthesiologist may be granted permission to use a computer terminal 10 to interrogate a barcode or other machine-readable code on a drug vial to extract the identity of the drug and print a label to be applied onto a syringe. The anesthesiologist can optionally also be granted permission to enter confirmation into the computer terminal 10, indicating that the interrogation of a barcode has returned the proper drug identification. The formulary and/or MDD entry corresponding to the now-confirmed drug can be updated by the anesthesiologist to indicate that the drug identified by the corresponding machine-readable code is accurate, and such confirmation can optionally be shared over the computer network 40 to at least one additional computer terminal. However, the anesthesiologist may be prevented from editing the formulary stored in the memory 24 of the computer terminal 10.

Additionally, an IT professional can be granted permission to address any technical, computer hardware-and-software-related issues with the computer terminals 10 that are unrelated to the specific drug information of the MDD and/or formulary. For example, the IT professional may be granted permission to assign and/or change: an IP address of the computer terminals 10, a security protocol employed, and other computer-specific matters. However, some information related to the formulary such as the version and description of the formulary can be viewed by the IT professional to ensure that the proper computer terminal 10 has the correct formulary installation. This also applies to version and description information of the operating system, BIOS 28, configuration data 32 and administration instructions 30.

The network 40 in FIG. 3 also includes an email server 46 through which email is to be transmitted to individuals who perform tasks related to the computer terminals 10 at the healthcare facility. The email server 46, like the computer terminals 10, and optionally other resources of the network 40, can transmit signals to other network resources via hard-wired communication channels (represented by solid lines 48 in FIG. 3) such as CAT-5 or CAT-6 Ethernet cable, via wireless communication channels (represented by arched, radiating signals 50), or a combination thereof. For example, email messages notifying individuals that a triggering event has occurred on one or more computer terminals 10 are transmitted from the email server 46 to one or more of the terminals 42, 44, a portable communication device 54 such as a personal digital assistant, cellular telephone, tablet or laptop computer, and the like. Additionally, the email server 46 can be configured to apply one or more rules that organize and deliver the information in more meaningful ways to the user. For example, a pharmacist may want notification of all problems with the formulary 36 (e.g., a “drug not found” notification) to be aggregated together and delivered to him at the start of his work shift and again 4 hours later. The email server 46 can be configured to transmit such notices in a single communication to the pharmacist at those times. Further, different pharmacists may prefer different notification procedures and different times at which such notifications are to be received, and the email server 46 can optionally be configured to satisfy the requests of each pharmacist individually. However, a group of IT technicians may want prompt notification of technical problems that prevent a computer terminal 10 from operating properly in a surgical suite. Again, the email server 46 can be configured to promptly transmit such notifications to the IT technicians substantially immediately upon detecting such technical problems.

Network resource allocation equipment 52 such as switches, routers, wireless access points, and the like can be included in the network 40 to share network resources and establish communication between the computer terminals 10 and the terminals 42, 44. Additionally, the computer terminals 10 can optionally serve as an expansion port to which other network resources such as the automated drug dispensing system 56, commonly referred to as a “smart cart”, can be connected to the network to dispense and document the strength, quantity and type of drug according to a schedule or in response to the occurrence of a predetermined event. Additionally, since one of the functions of smart carts is to control the dispensing of drugs and one of the functions of computer terminal 10 is producing labels for containers such as syringes that are filled with drugs from the smart cart, there are benefits related to efficiency if the devices can share information. For example, a network connection between the smart cart and computer terminal 10 will allow user login information such as username and password entered on one device to be shared with the other device so a user is authenticated on both devices with a single login. Other benefits include being able to share information about drugs being used in a procedure between the devices so verification and reconciliation of drugs can be performed to ensure the proper medications are dispensed, labeled and tracked for improving the accuracy of patient records and accurate billing. As shown in FIG. 3, the automated drug dispensing system 56 can be hard-wired to the computer terminal 10c, which is connected wirelessly to other network resources, but the automated drug dispensing system 56 can also optionally be configured to communication with the computer terminal 10c indirectly, through devices collectively making up the network 40.

As a specific example of the information shared between the computer terminal 10c and the smart cart 56 is drug consumption information. According to such an example, when information identifying a controlled substance (e.g., NDC) is entered into the smart cart 56 when such a controlled substance is to be removed and administered to a patient, information identifying that controlled substance can be transmitted to the computer terminal 10c. The transmitted information can be used by the computer terminal 10c to prepare and generate a label to be applied to a syringe acting as a delivery container for the controlled substance. However, the computer terminal 10c can also update a log stored in the memory 24 of drugs consumed in that specific OR in which the computer terminal 10c is located (and/or from that specific cart 56). For instance, when the controlled substance is accessed and obtained from the smart cart 56, the information identifying the drug entered to the smart cart 56 to unlock a secure drawer 57 (FIG. 3) of the cart 56 storing the controlled substance, or otherwise grant a clinician access to the drug is transmitted to the computer terminal 10c via the network 40 or via a local, hardwired connection. This information can be transmitted to the computer terminal 10c according to a predetermined workflow of the cart 56 and/or controller 81, or can optionally be transmitted to the computer terminal 10c in an effort to identify the drug that was not identifiable by the cart 56 (e.g., by the controller 81). At least a portion (but less than all), and optionally all of the information transmitted to the computer terminal 10c can be used by the computer terminal 10c to identify the drug and/or generate the label as described herein for labeling a syringe. Also based on the transmitted information, the computer terminal 10c can update a log in the memory 24 with consumption information that can be used to determine that a quantity of the controlled substance was removed from the smart cart 56. This consumed quantity can then be transmitted by the computer terminal 10c via the network 40 to the pharmacy terminal 42 or other suitable destination where inventory information for that OR is maintained (e.g., the controller 81 provided to the cart 56 as described below). Should the inventory of the controlled substance fall below a threshold value (e.g., the PAR value), the pharmacy terminal 42 can issue an alert to an appropriate party indicating that the controlled substance in the smart cart 56 should be replenished above the threshold value.

As noted above, however, medicinal substances that are not controlled substances (referred to hereinafter as “uncontrolled substances”) are often accessible from the smart cart 56 without entering access information identifying those uncontrolled substances as a condition for granting the clinician access to such drugs. The entry of such information is not typically mandated by or used to assist in the compliance with any regulations applying to controlled substances. However, a delivery container such as a syringe containing such uncontrolled substances is required by the law of a sovereign government or other governing-body regulations to be labeled. Thus, when such uncontrolled substances are removed from the smart cart 56, there may be no information to transmit to the computer terminal 10c if the clinician has elected not to voluntarily enter such information. However, when the computer terminal 10c is used to prepare and generate the label for an uncontrolled substance as described herein, the computer terminal 10c can update the log of consumption information stored in the memory 24 (or a computer-readable memory provided to the cart 56, for example) to reflect the consumption of the uncontrolled substance despite not receiving transmitted identifying information from the smart cart 56. Instead, the information obtained by the computer terminal 10c in response to using the scanner 18 to read a computer-readable code (e.g., NDC on the vial label) as described herein can be utilized to keep a running log of drug consumption based on the assumption that the drug being labeled was obtained from the cart 56. The computer terminal 10c can utilize the same or similar workflow when preparing labels for any uncontrolled substances that were not identified to the cart 56, as well as substances obtained from a so-called “dumb cart” that simply allows the manual retrieval of all drugs, including controlled substances, without entry of the drug identifying information to that cart. Thus, for smart carts 56 that lack an inventory capability for all drugs and dumb carts, the computer terminal 10c can allow for the maintenance, in real time as the drugs are consumed, or at designated periods of time that are allotted for the individuals tasked with maintaining the inventory of drugs in the carts, information pertaining to the remaining stock of drugs in such carts.

Although the embodiments above involve entering the NDC to identify the drug to the cart 56 and scanning a computer-readable code encoding the NDC with the scanner 18 to identify the drug, the present disclosure is not so limited. Alternate embodiments can entail entering any suitable information to uniquely identify a controlled substance to be accessed and removed from the cart 56 during a medical procedure, and reading any computer-readable code encoding any suitable information that allows a drug to be uniquely identified. For example, a proprietary code specific to the hospital or other health-care institution, private labeling standard, or other entity that is not subject to the control of a governmental or professional regulatory body can be used without departing from the scope of the present disclosure. An example of such alternate information or codes includes a hospital identification number (“HID”) used for internal purposes within a hospital or other healthcare facility. The HID can optionally be utilized elsewhere within the hospital or other facility to refer to the drugs in question, such as within an electronic medical record (“EMR”) system, billing system, and/or other system within the hospital or facility for purposes of managing the financial, business, and/or administrative aspects of providing healthcare.

The formulary 36 can also optionally include a field, value or other attribute for each drug or other substance having an entry in the formulary that indicates whether a label is to be printed for those respective entries. According to alternate embodiments, instead of a dedicated field indicating whether a label is to be printed, the memory 24 can store “null” values for the label information as a signal that a label is not to be printed for that entry, or any other suitable indication that, when referenced by the computer terminal 10c, instructs the computer terminal 10c to forego printing a label for the scanned substance. For example, eye drops may be administered to patients as part of certain medical procedures. However, eye drops that purely moisturize the eyes to minimize irritation of the eyes while the patient is sedated do not require a label to be printed and applied. Instead, the bottle of eye drops, which may already bear a label, is simply removed from the cart 56 and the eye drops administered to the patient. However, to aid in the monitoring of the cart's inventory, clinicians may be encouraged to scan a computer-readable code (e.g., barcode) on the bottle of eye drops before, after or during the medical procedure during which the eye drops are used. In response to reading this computer-readable code with the scanner 18 provided to the computer terminal 10c, the computer terminal 10c references the formulary 36, specifically the field indicating whether a label should be printed for this entry, to determine that a printed label is unnecessary for this substance. The computer terminal 10c creates, stores or updates the information evidencing consumption of the scanned eye drops logged in the memory 24 by the computer terminal 10c, but does not print or otherwise produce a hardcopy of the label as it does for labeling syringes containing other substances elsewhere herein.

In addition to the drug formulary 36, the memory 24 or other computer-readable medium accessible to the computer terminal 10c, locally and/or remotely over the network 40, can also optionally store another database with entries for non-drug items (referred to as “tools”) consumed or used in the OR where the computer terminal 10c is located. For example, syringes, gauze, intravenous lines, etc. may be stocked in the cart 56 and used during a medical procedure in the OR. As such, their supplies in the cart 56 must be replenished when they fall below a threshold level to ensure their availability during subsequent medical procedures in that OR. Unlike the formulary 36 of drugs storing NDC-compliant data for drugs subject to NDC rules and regulations, the tools having entries in the additional database can be any miscellaneous object other than a drug having an identifier assigned by a regulatory body, such as a NDC for example. Since the tools lack a NDC, the computer-readable code can be a UPC code, EAN code, or any other computer-readable code that uniquely identifies the tools. The computer terminal 10c can be configured with computer-executable instructions stored in the memory 24 to refer to this additional database when a computer-readable code is scanned to document the usage and/or consumption of the tools for purposes of monitoring the inventory of the cart 56. When the inventory of the tools available from the cart 56 falls below acceptable levels as defined by the facility or other party affiliated with the facility where the cart 56 is located, the log of inventory information transmitted by the computer terminal 10c can be used to determine what stocks need to be replenished, when, and the location of the cart 56.

Also, since the computer-readable code provided to, associated with, or otherwise used to identify the tools is not a NDC or other code compliant with a drug-labeling standard, the accuracy of such codes may not need to be verified as described herein to grant access to the tools in the cart 56. However, once the identity of the tool identified based on the scanning of the computer-readable code has been verified as accurate, such verification can be made available to one, a plurality, or each of the computer terminals 10 connected to the network 40. As an alternate embodiment, the verification of the accuracy of the tool identity based on the computer-readable code can be skipped at a time when the tool is accessed to be used during a medical procedure in the OR. The skipping of such verification can be recorded by the computer terminal 10c affiliated with the cart 56 so the identity of the tool can be revisited and later verified after a time when the tool is accessed for use. Again, verification can be shared over the network 40 for use by any of the connected computer terminals 10.

Embodiments of the computer terminal 10c can optionally combine and/or reconcile consumption data transmitted by the cart 56 and consumption data obtained by the computer terminal 10c in response to scanning a computer-readable code using the scanner 18 to arrive at a more-accurate inventory level in the cart 56. Thus, consumption of both controlled and uncontrolled substances can be accounted for. The inventory information indicative of the remaining drug stock can optionally be transmitted via the network 40 to a suitable destination where decisions regarding the replenishment of the drugs in the cart 56 can be made (for example, the pharmacy terminal 42, the controller 81 described below, etc.). Information pertaining to the updated log can optionally be transmitted by the computer terminal 10c in real time as the labels are generated, in batches such as following the conclusion of a surgical procedure or at the end of each day, or in any other desired manner to a desired destination to signal a need for drug replenishment.

The embodiment described above describes the computer terminal 10c maintaining an inventory of drugs stored by the cart 56. However, according to alternate embodiments, the inventory of drugs remaining in a cart can optionally be maintained by a computer terminal (e.g., the pharmacy terminal 42) remotely located from the computer terminal 10c, but accessible for communications from the computer terminal 10c over the network 40. For such embodiments, the consumption information can optionally be transmitted by the computer terminal 10c in real time as the labels are generated, in batches such as following the conclusion of a surgical procedure or at the end of each day, or in any other desired manner. The pharmacy terminal 42 or other recipient of the consumption information can be programmed to update the log of drugs consumed at a central location. The same, or additional log can optionally be updated for each of a plurality of computer terminals 10a, 10b, 10c located in different ORs, for example, and issue an alert when the remaining stock of a drug falls below a threshold value or provide information about the consumption of drugs upon request over the network 40 to a remote computer terminal such as the pharmacy terminal 42. In response to the issuance of such an alert, the pharmacist or other suitable party can replenish the drug(s) that are in short supply.

The alert issued to the pharmacist or other party who is responsible for replenishing the low-quantity drugs in a cart 56 can optionally include a replenishment confirmation option. Once an order for at least partial replenishment of the low-quantity drugs has been issued, the responsible party can select the appropriate replenishment option via a user interface presented by the pharmacy terminal 42, for example, indicating that a certain quantity of the low-quantity drug is to be replenished in the cart 56. The certain quantity can optionally be a predetermined number of single use vials to bring the number of vials in the inventory in excess of the minimum threshold quantity (e.g. PAR level), the quantity required to fully replenish the low-quantity drug(s), etc. Such a confirmation can optionally be transmitted for each low-quantity drug being replenished. When the replenishment confirmation is issued, the pharmacy computer 42 or other terminal from which such confirmation is sent can transmit information over the network 40 to the affected computer terminal(s) 10 or otherwise update the appropriate fields in the formulary 36 or other database (e.g., centrally maintained database for a computer terminal 10). This information can notify the affected computer terminal(s) 10 that the stock has been replenished so future drug consumption from the cart 56 can be accurately maintained by the corresponding computer terminal 10c.

According to alternate embodiments, the clinician replenishing the stock in the cart 56 can optionally manually enter the quantity of drugs being deposited in the cart 56 into the computer terminal 10c. Regardless of how the restocking information is conveyed to the computer terminal 10c or other database, a reporting component can be utilized to generate reports documenting the drug consumption and/or restocking information. For example, the reports can outline the quantity of drug(s) consumed and/or restocked, the locations where the drugs stored and require restocking, the times at which the drugs were consumed and/or restocked, the patients to whom the drugs were administered during a medical procedure, etc. for audit purposes.

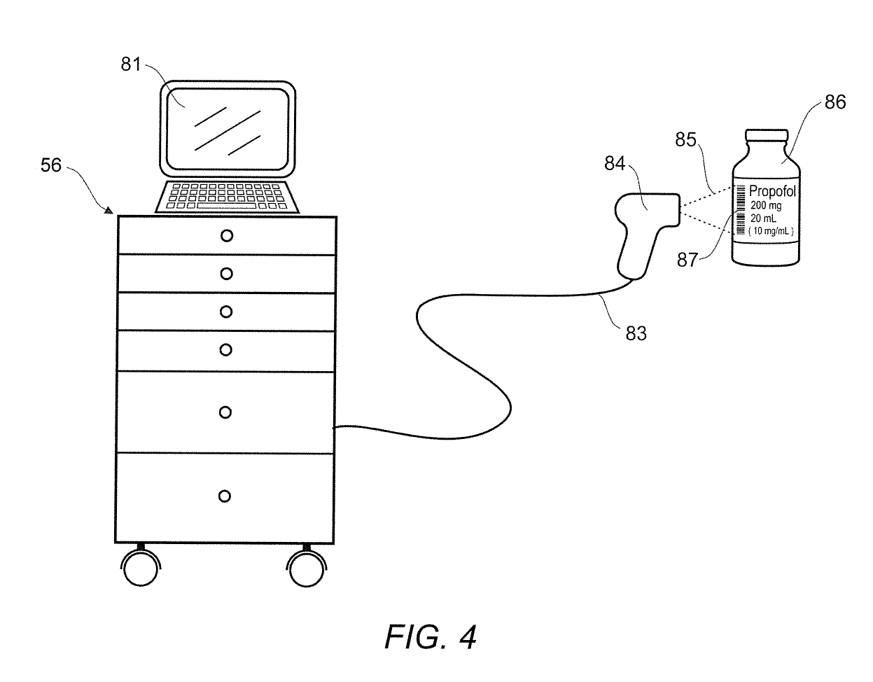

For many of the embodiments above, the information pertaining to the consumption of drugs and/or supplies from the drug cart is maintained in the memory 24 and/or another network-connected terminal such as the pharmacy terminal 42, for example. However, alternate embodiments of the drug cart 56, as shown in FIGS. 4-7, can optionally include a drug cart controller 81 dedicated, or at least specific to that respective cart 56. In other words, when the drug cart controller 81 is connected to the drug cart 56, either via a data cable such as a USB cable or docking station establishing direct communications with circuitry provided to the drug cart 56 or built into the circuitry provided to the drug cart 56 for example, the drug cart controller 81 maintains data concerning the contents and operation of the specific drug cart 56 to which it is provided. The drug cart controller 81 includes much of the same hardware as the computer terminal 10c.

For example, with reference to FIG. 2 for convenience, the drug cart controller 81 can be implemented as a computer including a processing component 22 provided to execute computer-executable instructions stored in a non-transitory, computer-readable memory 24 such as a hard disk drive, read-only memory (“ROM”), random access memory (“RAM”), optical disc, or any other suitable memory device, or any combination thereof, for performing the various functions described herein. The computer-executed instructions, when executed by the computer processor 22, result in the performance of the method of generating a label for a medicinal substance described in detail below. A BIOS 28 is provided to load the operating system and other such administrative instructions 30 stored in the memory 24 and manage hardware interface permissions of the computer terminal 10. The operating system can be configured to only load authorized updates to prevent unauthorized changes to the formulary 36, configuration data 32 and administration instructions 30. Configuration data 32 controls various features of the drug cart 56 (e.g., inventory database, security measures restricting access to drawers and/or specific drugs therein, etc.) that are active and available for use at any given time. A display 14 can also optionally be provided to the drug cart controller 81 to display information to a clinician during use, and a computer-input peripheral such as a non-contact scanner 18 can be provided to interrogate computer-readable codes. Although the internal components of the drug cart controller 81 are described with reference to FIG. 2, using the reference numerals appearing therein, any or all of the specific components can be configured specifically for use in managing the functions of the drug cart 56. For the sake of clarity, components such as the scanner are described and referenced hereinafter using the reference numerals appearing in FIGS. 4-7. Thus, in the description that follows, the scanner 18 is provided to the computer terminal 10c, while the scanner 84 is a hand-held barcode reader or other peripheral scanner provided to the drug cart 56. Embodiments of the drug cart 56 can include a drug cart controller 81 that lacks a hand-held scanner 84 entirely. For such embodiments, the scanner 18 provided to the computer terminal 10c can be utilized to provide such a drug cart 56 with the ability to track drugs dispensed and available inventory based on scans of barcodes 87 during the printing of labels based on the information encoded by those barcodes 87.

With reference to FIG. 4, a drug cart 56 can be configured to include a single hand-held scanner 84 in communication with the drug cart controller 81. Such drug carts 56 may have a single I/O port (e.g., USB port, wireless communication port, etc.) configured to receive a compatible data cable 83 and establish communications between only that single scanner 84 and the drug cart controller 81. The drug cart controller 81 is configured to receive and interpret data indicative of a barcode 87 or other computer-readable code applied to, or otherwise associated with a drug vial 86 removed from the drug cart 56 to identify the drug removed. Handheld scanners 84, however, may be configured with optics and barcode decoding algorithms optimized for reading a barcode on a flat surface, making it difficult to accurately read the barcode 87 applied to a curved surface such as the drug vial 86. Further, the vials 86 can be positioned at various different locations or orientations relative to the read zone 85 of the hand-held scanner 84 each time a barcode 87 is scanned making it more difficult to produce a successful scan. For example, the read zone 85 of the hand-held scanner 84 may encompass a relatively-small area of the vial 86, whereas the scanner 18 provided to the computer terminal 10c includes optics that enlarge the read zone 95 of the scanner 18 to encompass a relatively-large portion of the vial 86. However, the computer terminal 10c can optionally include computer executable instructions or otherwise be configured to correct an optical view of the barcode 87 extending about a curved surface of the vial 86. Additionally, since the scanner 18 of the computer terminal 10c is integrated into the bottom of the display 14 above an opposing portion of the housing 20, the limited space between the scanner 18 and the housing 20 confines the vials 86 to a small region in which the scanner 18 is focused to read barcodes 87. The substantially fixed scanning distance and designated area for resting vial 86 during the scanning process on terminal 10c further enhances the speed and reliability of the scanner to successfully decode barcode 87 because of the predictable location of the barcode 87 relative to the scanner 18. Such design enhancements improve the readability of the barcode 87, thereby making the scanner 18 of the computer terminal 10c more forgiving to the orientation, physical shape and/or position of the vial 86, and more user friendly thereby encouraging compliance by users to scan drug vials. But since the drug cart 56 is typically configured to connect with a single data cable 83, the drug cart 56 may lack the option for connecting a second, different scanner to be used for interrogating the barcode 87 to identify a drug removed from the drug cart 56. And even if a replacement scanner can be installed in place of the hand-held scanner 84, the drug cart controller 81 may not be configured to properly interpret the signals transmitted by such a replacement scanner.

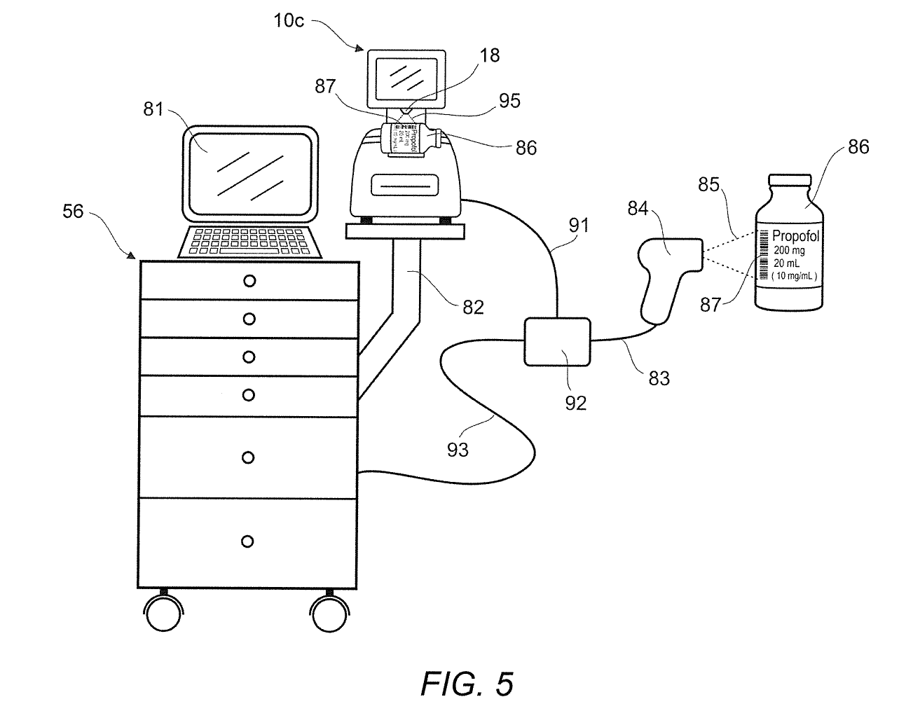

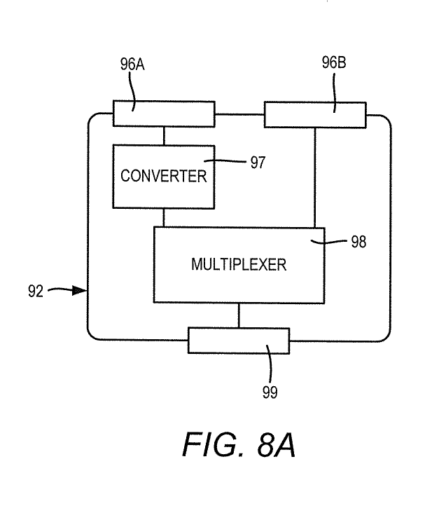

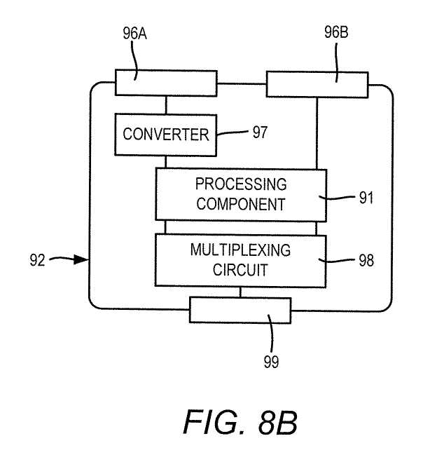

As shown in FIG. 5, the drug cart 56 further includes a shelf 82 on which the computer terminal 10c can be placed, and a multiplexer 92 that allows at least the computer terminal 10c to be operatively connected to the drug cart controller 81, in addition to the hand-held scanner 84. An embodiment of the multiplexer 92, shown in FIG. 8, includes a plurality of input ports 96A, 96B to which the computer terminal 10c and the hand-held scanner 84 can be connected, respectively. Although two input ports 96A, 96B are shown, any desired number of ports can be added in accordance with the present disclosure. A multiplexing circuit 98 coordinates communication of signals via each of the input pots 96A, 96B to be output over a single output port 99, which is connected to the drug cart and/or drug cart controller 81 via a data cable 93 (FIG. 5).

The multiplexer 92 can be configured to receive data on an input port 96A that is formatted in accordance with a data encoding specification. Input ports 96A, 96B can each optionally be individually configured to receive data formatted in accordance with different, or the same, encoding specifications. Input ports 96A, 96B can each optionally be configured to automatically recognize the encoding specification of the received data. For example, the scanner 84 can be configured as a so-called “plug-and-play” peripheral that is recognizable in response to being plugged in, and without separate user interaction.

The input port 96A to which the computer terminal 10c is to be connected can communicate with an optional converter 97 that converts content transmitted by the computer terminal 10c into content that can be properly interpreted by the drug cart controller 81. The converter 97 can, for example, include a computer processor programmed with computer-executable instructions defining an algorithm for altering or translating the content of a transmission by the computer terminal 10c into a state that can be processed and interpreted by the drug cart controller 81. For instance, the converter 97 can format data transmitted by the computer terminal 10c in response to scanning the barcode 87 during a process for printing a label for a syringe that is to contain the respective drug. As another example, the converter 97 can extract a certain subset of information included in the transmission by the computer terminal 10c to be relayed to the drug cart controller 81, optionally in a format the drug cart controller 81 is configured to interpret or at least process. Regardless of the data extracted in response to scanning the barcode 87, the converter 97 can translate or otherwise modify that data in compliance with a format, standard, etc. of data received from the hand-held scanner 84. Thus, the computer terminal 10c can optionally transmit data consistent with a first format, language, modulation protocol, etc., that is not understood by the drug cart controller 81, and the converter 97 can render that data into a second, different format, language, modulation protocol, etc. that can be processed and interpreted by the drug cart controller 81, optionally without modification of the drug cart controller 81 for this specific purpose. Accordingly, the multiplexer 91 connected to the computer terminal 10c emulates the hand-held scanner 84 recognized by the drug cart controller 81, facilitating communications between the computer terminal 10c and the drug cart controller 81 that would otherwise not occur due to incompatibilities between the computer terminal 10c and the drug cart controller 81. The drug cart controller 81 can then process data received in a communication from the computer terminal 10c in the same manner the drug cart controller 81 would have processed data obtained received from the hand-held scanner 84.

The multiplexer 92 can optionally be configured with a processing component 91 that includes a computer processor, a non-transitory, computer-readable memory unit containing computer-executable instructions (e.g. ROM) and a transitory, computer-readable/writable memory (e.g. RAM) unit for storing, retrieving and manipulating data. The computer processor can execute computer-executable instructions stored in a non-transitory, computer-readable memory. The computer-executed instructions, when executed by the computer processor, result in the performance of a method to store the data received by ports 96A, 96B from both the hand-held scanner 84 and scanner 18 into RAM and analyze and translation of the data. That data can optionally include additional timing data indicative of the relative or absolute time of when the scan data was received. If both ports 96A, 96B receive scan data identified by the computer processor as the same barcode 87 from vial 86 within a time frame that is configured on the multiplexer 92, this condition may be indicative of the same drug vial 86 being scanned twice. The processing component 91 can be configured to pass none, one or both of the scan data occurrences identified as being from the same drug vial 86 to the output port 99. Additionally, the multiplexer 92 can optionally be configured to receive information from the drug cart controller 81 using the output port 99 when such port can by configured for bi-directional communications, that indicates the status of the medical procedure that cart 56 is engaged in. One such example of this status information would be the start and stop of the medical procedure. The status information received from the cart 56 can be used in the analysis of scan data by the computer processor to improve the accuracy of identifying multiple drug scans that may be from the same drug vial 86 and therefore should be reported as single drug scanning events.

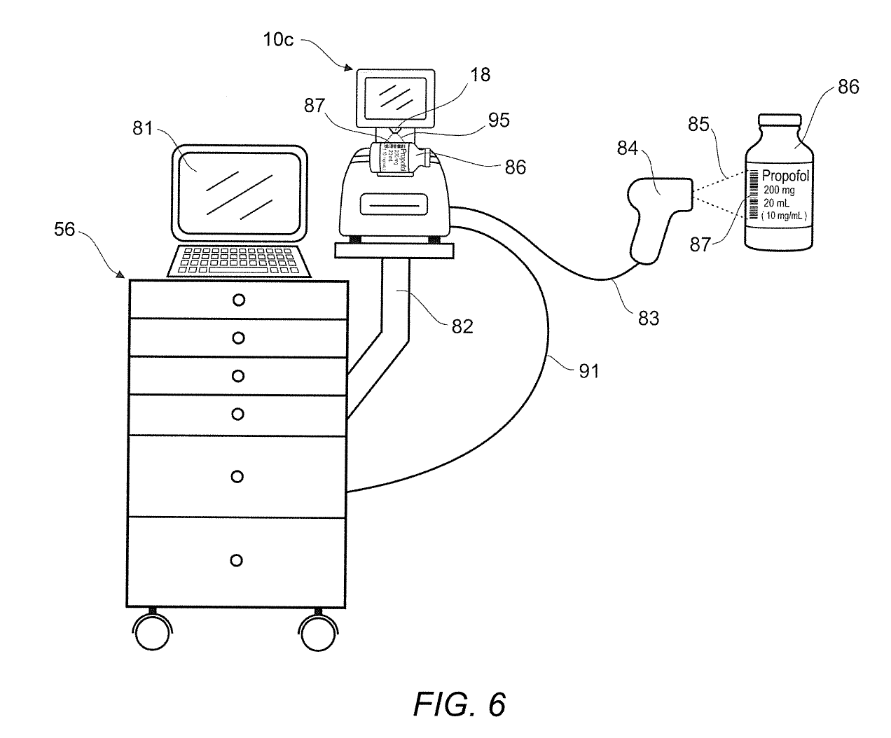

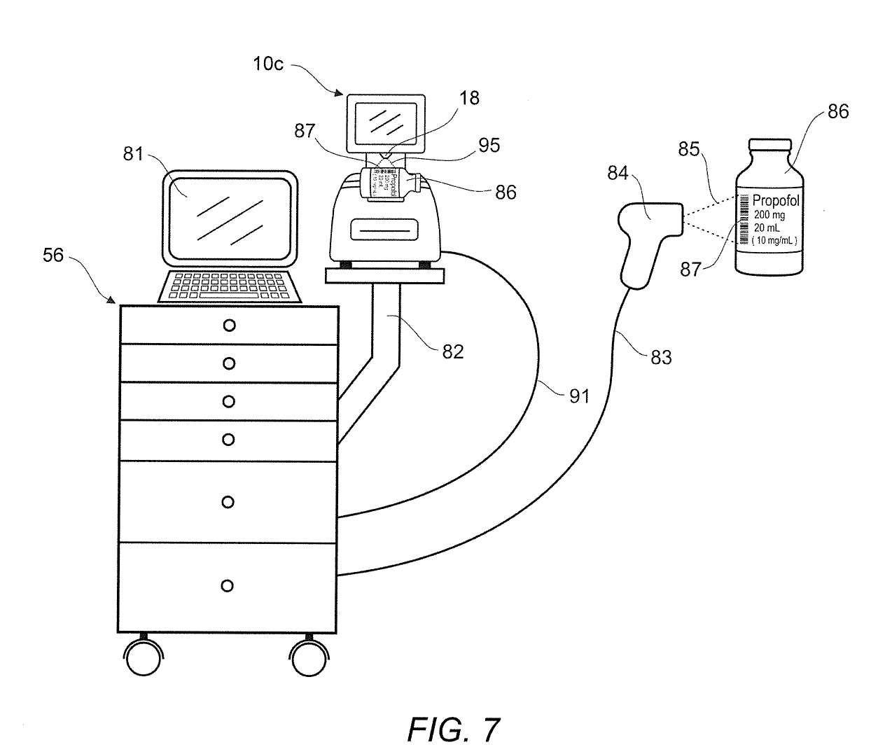

The multiplexer 92 can be an add-on, peripheral component with its own housing 100, separate from and external to the drug cart controller 81 and the computer terminal 10c, as shown in FIG. 5. Alternate embodiments of the multiplexer 92 can be integrated into the computer terminal 10c as shown in FIG. 6. The embodiments of FIGS. 5 and 6 can provide a drug cart controller 81 and/or a drug cart 56 configured to receive signals indicative of a computer-readable code from a single source with an ability to receive such signals from the computer terminal 10c. In other words, the multiplexer 92 simulates a single scanner connected to the drug cart controller 81. Additionally, the embodiment of FIG. 6 utilizes the computer terminal 10c as an intermediary, through which data obtained in response to reading the barcode 87 using either the hand-held scanner 84 or the scanner 18 of the computer terminal 10c is conveyed. In addition to eventually being used by the drug cart controller 81 to document consumption of the drug cart contents, at least a portion of such information can be utilized by the computer terminal 10c to print a label for the drug or other supply encoded with the barcode 87 as described herein. For example, different data obtained by scanning the same barcode 87 may be used by the drug cart controller 81 and the computer terminal 10c. According to yet other embodiments, the multiplexer 92, or at least the components giving rise to its functionality, can be integrated into the drug cart controller 81 and/or drug cart 56 itself, as shown in FIG. 7. The multiplexer 92 can also optionally be configured to transmit data obtained using the hand-held scanner 84 to the computer terminal 10c, without instructions to do so from the drug cart controller 81. Additional embodiments of the drug cart controller 81 can include a plurality of USB ports or other input ports and an operating system that supports concurrent support of the scanner 18 provided to the computer terminal 10c and the hand-held scanner 84.

Regardless of the embodiment, the computer terminal 10c can transmit at least a portion of the information obtained in response to reading the barcode 87 or other computer-readable code to the drug cart controller 81 for documenting the consumption of a drug or other supply obtained from the drug cart 56. In use, a clinician who will administer a drug from the drug cart 56 logs into the drug cart 56 by entering information that can be used to validate the clinician's authorization by a healthcare facility where the drug cart 56 is located to access the contents of one of the drawers, including the drug. Once the drug has been obtained from the drug cart 56, the clinician may not be inclined to use the hand-held scanner 84 to read the barcode 87 and enter the drug information into the drug cart controller 81, which may be required solely for purpose of tracking the consumption of the cart's contents. In other words, a clinician whose primary concern is treating the patient may not feel compelled to track drug consumption from the drug cart 56, and the healthcare facility may not mandate drug consumption tracking by clinicians, instead putting that burden on the pharmacy. However, labeling drugs to be administered to patients can be mandated by one or more of a government regulation, professional organization, internal healthcare facility policy, and the like. Since using the computer terminal 10c to generate a label 12 compliant with such regulations lessens the labeling burden on the clinician, the clinician will initiate the process of printing a label 12 using the computer terminal 10c.

During the process of printing a label for a syringe or other delivery container for the retrieved drug, the clinician scans the barcode 87 applied to the vial 86 using the scanner 18 provided to the computer terminal 10c. In response, the computer terminal 10c references the formulary 36 (FIG. 2) to identify at least one of, a plurality of, or all of the NDC, the drug name, drug concentration (or total dose and/or total volume contained in the vial 86), and the HID. At least a portion of this information is included, optionally with additional content such as expiration date, preparer's name or identification, and patient identifier for example, by the computer terminal 10c among label content to be printed by the printer 26 of the computer terminal 10c onto label stock for labeling the syringe. Also in response to scanning the barcode 87, however, the computer terminal 10c transmits a portion of the information obtained from the formulary 36 based on the barcode 87, and optionally the information indicative of the barcode 87 itself, to the drug cart controller 81. When received, this information can be stored in the memory of the drug cart controller 81, optionally supplemented with additional information, for documenting the consumption of the drug cart's contents.

In some cases, such as restocking drugs into cart 56 for example, the normal workflow of scanning a barcode 87 affixed to drug vial 86, printing labels for syringes on terminal 10c and decrementing drug inventory levels on cart 56 does not apply. Instead, barcodes 87 scanned represent vials 86 of that drug being added to the cart, not removed, so no labels 12 are required to be printed and the inventory levels should be increased, not decreased. To accommodate such conditions, it is useful to provide a method for the cart 56 and terminal 10c to enter a different mode of operation in response to a user initiated restocking activity on the cart 56. According to one embodiment, the clinician can input an instruction using the touchscreen display 14 of the computer terminal 10c to place the computer terminal 10c in a restocking mode. In the restocking mode, the computer terminal 10c can optionally transmit a signal to the drug cart 56 to notify the drug cart controller 81 of the drug cart 56 that drugs are being stocked. Thus, the scanner 18 of the computer terminal 10c provided to a drug cart 56 that lacks a hand-held scanner 84 can be used to scan the barcode 87 on a vial 86 of a drug being added to the cart 56, and a quantity of the vials being added entered via the touchscreen display 14. Optionally, the computer terminal 10c can be configured to allow barcode 87 on a vial 86 to be scanned on each individual as the vial 86 is added to the cart 56 indicating a quantity of one drug is being added, avoiding the need to manually enter the quantity of the vials added via the touchscreen display 14. The drug information, and the associated quantity added is transmitted to the drug cart controller 81 to establish the stocked quantity. While in the restocking mode, the computer terminal 10c can be configured not to print the label 12 for the drug in response to scanning the barcode 87 as described elsewhere herein.

In another embodiment of the invention, entry of the restocking mode can be input into the drug cart controller 81 of the drug cart 56. In response, the drug cart controller 81 can transmit a signal to terminal 10c indicative of the restocking mode, or other certain modes of operation, on the cart 56 that was initiated by the user via the drug cart controller 81 instead of the computer terminal 10c. Terminal 10c is configured to receive the signal and perform a specific set of operations associated with the specific signal. For example, if a pharmacy technician logs into cart 56 at night to restock drugs used during the day, the restocking procedure may involve scanning the barcode 87 on each group of drugs 86 as they are placed in the cart 56. In this circumstance, it is not necessary to print labels on terminal 10c for use on syringes as the barcodes 87 of drugs being restocked are read, yet the scanner 18 or the hand-held scanner 84 are used by the pharmacy technician to scan the barcode 87. To handle this workflow, the cart 56 transmits a signal to terminal 10c when the restocking operation is initiated by the pharmacy technician via the drug cart controller 81. In response to receiving such a signal, the operation of terminal 10c is altered and label printing is disabled for each barcode 87 that is scanned during restocking. According to an alternative embodiment, rather than disabling printing of labels 12 by the computer terminal 10c, the drug cart controller 81 can optionally be configured to not transmit to the computer terminal 10c data indicative of, or encoded by the scanned barcode 87, in response to scanning barcodes 87 using the hand-held scanner 84 provided to the drug cart 56 during restocking.

Alternately, terminal 10c can directly support other operational modes based on the permissions of specific users accessing the device. Users such as pharmacy technicians, for example, can have permission granted in terminal 10c when they log in to read a barcode 87 using scanner 18 or hand-held scanner 84 and transmit the data to cart 56 but have no permission to have terminal 10c print a label.

In yet another embodiment of the invention, terminal 10c can be configured with a default operational mode requiring no log in that is different from the operation mode when a user is logged in. For example, the device can permit a user to read a barcode 87 using scanner 18 or hand-held scanner 84 without logging in and transmit the data to cart 56 without printing a label.

In another embodiment of the invention, the drug cart 56 can transmit patient information to the computer terminal 10c over the network 40 related to the drugs being prepared for the patient. At least a part of the patient information received by computer terminal 10c is stored in memory 24 and is associated with the drugs that are being prepared on computer terminal 10c. The combination of drug information and associated patient information can be transmitted by the computer terminal 10c via the network 40 to the pharmacy terminal 42 or other suitable destination where inventory information for that OR is maintained. An interface component on pharmacy terminal 42 can be utilized to generate information in a suitable format for use by an EMR or anesthesia information management system (“AIMS”) 77, for example and transmit the information over network 40 about the drugs used for a specific patient. For example, the interface component can transmit messages to the AIMS 77 indicating that the drugs Propofol and Fentanyl with the corresponding NDC or HID values were used on patient with ID 123456789 and optionally include the location they were used such as operating room 3. This information can be used by the AIMS 77 system as a part of its normal function and processing, and optionally the information can be incorporated into the patient medical record by the AIMS 77. Alternately, the pharmacy terminal 42 can transmit the same information to the EMR in a suitable format that adds the drug usage information directly to the specific patient's medical record on the EMR system. This combination of patient information associated with drug information can ensure proper patient medical records, drug charge capture and billing for business purposes, drug tracking to assist in determining the amount of a drug that was used on a patient and the amount that was not used based on the original drug amount of the drug prepared as reported by computer terminal 10c for monitoring drug waste management. Additionally, the information may be used for general inventory management of drugs.

Before the computer terminals 10 are usable in a medical environment, the AT software can be installed on one or more of the terminals 42, 44 to be used by a pharmacist at a hospital or other health care facility to populate the MDD. The AT accepts drug information from various sources such as commercially available drug databases (e.g. Lexicomp) and allows the pharmacist to selectively add drugs to the MDD, which can be stored at network-accessible storage server or locally by the terminal 42, 44 running the AT. In simplest terms, the MDD is the set of drugs available to the hospital or other healthcare facility.

Once the MDD is populated with drug information, the pharmacist will use the AT to select a subset of drugs from the MDD to be added to the formulary that will be stored in the memory of one or more of the computer terminals 10, thereby enabling the computer terminals 10 to recognize the drugs in that formulary. The formulary managed using the AT running on one of the terminals 42, 44, as it pertains to the computer terminals 10, can be considered an official set of medications with associated information for preparing and labeling drug containers in accordance with a medical labeling standard. The “associated information” can include information for preparing the drug, which usually means diluting the drug when needed. It can also include information related to the color, patterns, graphics and textual information printed on the label for specific drugs to render those labels, once printed, compliant with the medical labeling standard. Other types of associated information can be files, data for implementing a computer-generated voice, references to files for audibly pronouncing the name of the drug and important drug related information such as the concentration value and concentration units, or any combination thereof. For example, in the case of Propofol 10 mg/ml, a single audio file, or more than one audio file or references to audio files can be combined together to audibly speak the drug name and concentration of the drug as “Propofol ten milligrams per milliliter”. According to the present example, the drug name “Propofol” can be contained in one audible file while the concentration value “ten” is in another audible file and the concentration units “milligrams per milliliter” in a third audible file. These three audio files can be executed and played in sequence to allow the computer terminal to audibly broadcast “Propofol ten milligrams per milliliter” via the speaker 17 in response to the scanning of a barcode associated with the container that contains 10 mg/ml Propofol. Other audible information including information about errors such as “do not use drug”, for example, can also be associated with a drug in the formulary using the AT. The “do not use drug” audible information can optionally be audibly output using the speaker 17 when a drug has been recalled and a pharmacist wants the computer terminals 10 to notify users not to use a drug that has been recalled, or is otherwise not suitable for use, for example. The computer terminal 10 can automatically assign some audible drug information by examination of the data related to the drug. For example, the concentration value 10 can be used to select the audible file or file reference that speaks the word “ten”. The same applies to the concentration units. mg/ml can automatically be used to select the audible file or file reference corresponding to “milligrams per milliliter”. Since the MDD can include information on many types of drugs used in the hospital including pills, capsules, ointments, patches, injectables, etc., the pharmacist can optionally select only the drugs from the MDD that are commonly used by anesthesiologists in the operating room (interchangeably referred to herein as the “OR”) for a particular procedure or other points of care in the facility where drug containers are labeled prior to dispensing to patients. These are usually the injectable drugs. This subset of drugs can optionally be further narrowed into application-specific sets for pediatrics, etc. . . . .

Once the pharmacist selects the drugs for the formulary and assigns the associated information to each drug, a formulary “package” is created. This package is a single electronic file containing all formulary information in a format suitable for delivery to the computer terminals 10 on which the formulary is to be stored. Assembling the formulary into a single package simplifies the transfer of information from the terminal operating the AT to the intended computer terminals 10. It also ensures that all information for that version of the formulary to be transferred to the computer terminals 10 is encapsulated in a single file so no information is lost or forgotten. The formulary package is then transmitted over the network 40 to the computer terminals 10 intended to receive the formulary package, as selected using the AT. According to alternate embodiments, the formulary package can optionally be stored on a USB flash drive and delivered to the computer terminals 10 by plugging the USB flash drive into the computer terminals 10 that are to receive the formulary package, which is then automatically installed. This makes the transfer an all-or-nothing proposition, meaning that the existing formulary on the computer terminals 10 is completely replaced by the formulary package being transferred. If the received formulary package is incomplete or corrupt, it will not be able to be installed on the computer terminals 10, and the user will be alerted to the installation failure.

In addition to delivering formulary packages, the computer terminals 10 accept other types of packages for configuration and software updates. Any of these packages can be delivered via USB drive or network. All packages are encoded with a digital signature to prevent the contents of the package being altered or corrupted. Additionally, the USB flash drive can optionally be required to possess a predetermined digital signature to ensure that only authorized USB flash drives can be plugged into the computer terminals 10 to install a formulary, configuration or software update package.

For example, a configuration package 32 stored in the memory of the computer terminals 10 controls the behavior of those computer terminals 10 when preparing and labeling syringes. It is can be used to enable or disable features of the computer terminals 10 such as requiring verification that the drug information displayed on touch-screen display 14 matches the drug container scanned by scanner 18 before printing the label. A pharmacist, head of anesthesia or other authorized individual can customize the workflow to dictate how syringe preparation will be handled and use the configuration package to cause the computer terminals 10 to conform to that desired. Once the configuration package is installed, the computer terminals 10 can impose that workflow on the user (e.g., requiring an authorized confirmation). Multiple workflows can be installed on any given computer terminal 10. In some cases, a user can be granted permission to select a workflow for their use on computer terminal 10. A workflow can optionally be selected based on a user's login information. This allows different workflows for different users. For example, a new resident in the anesthesia program may have all extra verification enabled while a senior physician may have a different workflow configuration. Each workflow can define a sequence of actions to be performed, and optionally is required to be performed, by a user when interacting with the computer terminals 10.

From time to time the software such as the operating system on the computer terminals 10 may need to be updated and/or upgraded. A software update package from a proprietor of the computer terminals 10 may be created and transmitted on a USB flash drive, CD, and/or over a communication network to a hospital for installation on the computer terminals 10, which may change or improve the operation of the system.

Each formulary, configuration and software update package has an identifier string and version number. The identifier can provide human readable information that describes the contents of the package (e.g. Pediatric formulary). A unique version number is assigned to formularies and configuration packages automatically by the AT or from the vendor in the case of software update packages. The combination of the identifier string and version number makes each package easy to identify and track. The computer terminals 10 can display this information on the touch-screen display 14 or send it over the network 40 for remote monitoring. This is useful for tracking which systems have been updated and which system have not.

As described above, a plurality of different formularies may be needed for different purposes. One formulary may contain drugs for general adult surgeries while another may contain different drugs or preparations (dilutions) for pediatric procedures. The AT allows multiple formularies to be created and managed from a single MDD. The user interface of the AT that controls the deployment of formulary packages over the network 40 allows the user to select a single computer terminal 10, as might be required for testing a new version of a formulary before wide-scale deployment, or a plurality or all of the computer terminals 10. In the case of multiple computer terminals 10, these can be manually selected or pre-assigned in groups so all computer terminals 10 in a group can receive the same formulary.

Related to the installation of packages such as formularies, a distribution list of authorized computer terminals 10 can optionally be encoded with the formulary package or other packages such as the configuration package or software update package. The distribution list defines which computer terminals 10 are allowed to install the package. A computer terminal 10 checks the distribution list before installing the package to see if it is on the list. If the computer terminal 10 is not on the distribution list, the package will not be installed. In other words, rather than individually selecting the computer terminals 10 using the AT to which the package is to be transmitted, the computer terminals 10 that are intended to receive each package can be included in the distribution list in the packages themselves. The packages can then be transmitted via the network to all computer terminals 10, but installed on only those computer terminals 10 included on the distribution list. This is particularly useful when a facility uses USB flash drives to distribute packages, but can also apply to network installed packages. For example, a USB flash drive containing a formulary package for general adult surgery might be inadvertently be plugged into a computer terminal 10 intended for pediatric use. The distribution list embedded in the package prevents the pediatric computer terminal 10 from installing the formulary package for the general adult surgery onto the computer terminal 10 intended for pediatric use.

Each computer terminal 10 can optionally be limited to store a single formulary at a time, but alternate embodiments can allow a plurality of different formularies to be installed and selected by the user as the user logs into the computer terminal 10. Alternately, a formulary could be tied to, and automatically selected as the active formulary based on the login information of the user when that user logs in. This would allow a Gastroenterologist, for example, to recognize a different set of drugs with the computer terminals 10 for minor procedures than an anesthesiologist for general surgeries.

In another embodiment, a single formulary 36 can contain drug information suitable for multiple types of procedures such as pediatric, cardiac, general surgery, gastroenterology, minimally invasive surgery and others in a single formulary. The user of computer terminal 10 can select the type of procedure being performed. The type of procedure selected would correspond to a specific subset of drugs and associated drug information contained in formulary 36. For example, a specific drug may not require dilution when used in typical adult surgeries, but may require dilution in pediatric procedures. A single formulary can have different information for preparing the same drug based on the type of procedure currently selected. Additionally, configuration data 32 can be used to limit the procedure types available to a particular user. For example, an anesthesiologist may have full access to all procedures, but a gastroenterologist may be limited to drugs suitable for procedures such as colonoscopies.

Related to a single formulary containing drug information for multiple types of procedures, a default selection of the procedure type can be made based on the user login information on computer terminal 10.

When packages are deployed to the computer terminals 10 over the network, options can be specified that determine when the packages will be installed. It is undesirable to cause a package to be installed in the middle of a medical procedure, so options to defer package installation until the user logs out of the computer terminal 10, or after a specific time, such as 10 PM, or a combination of options such as the first time no user is logged in to the computer terminal 10 and the time is after 10 PM. Other options such as install on next reboot are also possibilities. An optional time delay can be specified that will not immediately install a package when a user logs out. This is to handle the case where one physician goes on break during a long procedure and another physician fills in for the physician on break. In this case, a logout may be followed by another login because the procedure is still underway. A reasonable delay is needed to ensure another user is not going to login. This can also be accomplished by displaying a warning message on the touch-screen display 14 that a package is about to install and a delay to allow the user to touch the screen to defer the installation, providing enough time and notification for the user to log into the computer terminal 10.

Each computer terminal 10 is designed to operate autonomously. Once it is has a formulary and configuration package installed, the computer terminals 10 will operate with or without a network connection. This ensures the device will continue to work and not interfere with the medical procedure even if the network connection stops functioning. While the network is not functioning the computer terminals 10 will store information that needs to be transmitted for logging, record keeping, billing, and other purposes when the network connection is re-established.

When the computer terminals 10 are connected to the network 40 and the network connection is functioning properly, they can perform other functions in addition to receiving packages. For example, the computer terminals 10 can transmit information regarding the status of the: hardware (e.g., the printer 26 is low or out of a particular printing ink or toner, the printer is out of label stock), package information such as versions of packages installed, the user logged into each of the computer terminals 10 (if any), important events such as “drug not found” alert in response to scanning a barcode with the scanner 18, for example, which may indicate a drug is in the hospital that was not included in the formulary on that computer terminal 10 and may not be properly usable, etc. In such situations, an alert signal is transmitted by the afflicted computer terminal(s) 10 to the email server 46, and the email server 46 responds by composing the email or other message containing textual information corresponding to the alert signal and transmitting the email or other message to the intended recipient. The status information can optionally be transmitted by the computer terminals 10 automatically, not in response to receiving a status request, upon the occurrence of an event, periodically, when a status changes, or a combination thereof. According to an alternate embodiment, the AT running on the terminal 42 or 44 can be used to access the computer terminals 10 over the network 40 to determine the status of each computer terminal 10, the various components making up the computer terminals 10, or other information regarding the computer terminal 10. Thus, the AT running on the terminal 42 or 44 can be used to receive status report information autonomously transmitted by the computer terminals 10, and/or can be used to retrieve (or request transmittal of) the status report information from the computer terminals 10. The status report information can optionally be tabulated by the AT running on the terminal 42 or 44 and presented in a logical manner to the user, thereby allowing the user to readily identify any of the computer terminals 10 that are not operating as intended.

In another embodiment, event information that occurs on a computer terminal 10 can be shared with other computer terminals 10 on the network 40 either through the AT running on one or more of the terminals 42, 44, or the email server 46, or with a dedicated software program on the network 42, or directly with other computer terminals 10 on the network 42. Shared information can be used to optimize the workflow of the users by sharing events such as first time verification of a drug being used at a computer terminal 10 so other users will have the benefit of the drug verification and not have to perform the same verification procedure on each computer terminal 10.

Related to the aforementioned sharing of information between computer terminals 10 on the network 40, a syringe or other container labeled by the computer terminal 10 can include a unique identifier in a machine-readable format on the label. For example, a unique serial number could be assigned to each syringe and encoded in a barcode that is applied to the syringe. Information related to the unique identifier numbers of the containers prepared at a particular computer terminal 10 and information about the drug in the container (e.g., drug name, concentration, expiration date and/or time, other information, and any combination thereof) can be shared with other computer terminals 10 on the network 40 so a container that is prepared for one patient but is moved to another operating room can be verified when the machine-readable code is scanned by the scanner of the computer terminal 10 in the other operating room. As a result of scanning the barcode or other machine-readable code, a notification can be provided to the user, alerting the user that the drug within that drug container is not intended for that patient (i.e., it is intended for the patient in the original operating room). Alternately, for drug containers permitted to be moved between operating rooms, the contents of the container can be verified in each operating room, and whether the contents are expired, by scanning the machine-readable code with the scanner 18 provided in each of those operating rooms.

Messages of importance to users such planned updates to software, formularies, configuration changes or even messages such as staff meetings or departmental messages can be sent out over the network 40 from an AT running on terminals 42, 44 to one or more computer terminal 10 systems on the network and displayed on the touch-screen display 14 when the user logs into the system. If the message is received on a computer terminal 10 while a user is logged in, a non-intrusive message will notify the user that a message is waiting to be displayed. This will prevent any interruption of the user in the middle of a medical procedure. Messages can be configured to display once per user or each time a user logs in until the message is discontinued from the terminal(s) 42, 44 running the AT. Authority to send out or discontinue messages can optionally be granted or restricted to specific users of the AT.

The usefulness and effectiveness of computer terminal 10 can be enhanced by associating patient information with a medical procedure. Patient information at a healthcare facility is usually stored on an EMR. The EMR typically collects and manages patient Personal Health Records (PHR) from sources throughout the healthcare facility and makes those records available to authorized users and equipment through the network. As related to computer terminals 10, patient information can be transmitted over the network 40 to one or more of the computer terminals 10 from an EMR system in the healthcare facility using HL7 or another healthcare specific network protocol. Patient information such as patient name, ID, date of birth, sex, medical conditions, drug history and other relevant information from the EMR is received and stored by an EMR gateway server. The EMR gateway server can collect and aggregate patient information received from the EMR when the EMR transmits information over the network 40. In other words, the EMR gateway server receives information such as ADT (admission-discharge-transfer) codes and other HL7 messages transmitted from the EMR to different devices intended for different recipients over the network 40. Each such transmission from a plurality of different EMR servers can optionally be collected and recorded by one common gateway server or a plurality of gateway servers. Thus, the information collected by the EMR gateway server can be accessed and retrieved from the EMR gateway server rather than from the EMR server. Since patient information is often transmitted on the network from the EMR as it becomes available from different sources in the healthcare facility, it is necessary to collect and combine the patient information so the appropriate information it is available for a specific purpose. The EMR gateway server performs this function for the computer terminals 10. The EMR gateway server can also reduce the cost of connectivity to the EMR because many EMR systems have a fee per connection and it can be less expensive to connect one EMR gateway server to the EMR than many individual computer terminal 10 systems. An EMR, an EMR in combination with an EMR gateway server, and a plurality of EMR systems in network communication with a common EMR gateway server are represented generally at 47 in FIG. 3. Patient information is transmitted from the EMR gateway server 47 on network 40 to computer terminals 10 when a specific patient identity is entered into a computer terminal 10 as the patient that is to receive medical attention at a location, such as in an operating room of a healthcare facility for example, where the computer terminal 10 is located. The specific patient's identity can be selected from a patient list stored by the EMR gateway server 47 and accessed using the computer terminal 10, or determined in response to the user entering unique patient identification information such as a patient ID using touch-screen display 14 or scanner 18, for example. The patient ID or other patient-related information can be transmitted over the network and used to look up the patient information from the EMR gateway server. The patient information received from the EMR gateway server by computer terminal 10 is verified by the user using touch-screen display 14 and stored in memory 24 for the duration of the procedure. Likewise, the user can enter, or select from a list displayed via the display 14, the specific procedure to be performed, which is stored in the memory 24 and associated with the patient information. The procedure can optionally also be transmitted from the computer terminal 10 over the network 40 to be stored in association with an entry for that patient in the EMR gateway.

Patient information related to drug allergies, other drugs the patient is taking and relevant information such as date of birth, sex, weight, etc. can affect the selection of medications and doses administered during a medical procedure. Patient information can be associated with a procedure on computer terminal 10 as described above. In the simplest use case, the patient information locally stored in memory 24 on the computer terminal 10 can be displayed on touch-screen display 14 for review by the user. In more complex implementations, the patient information in memory 24 can be accessed by the processing component 22 of the computer terminal 10 and checked as drugs are being prepared on computer terminal 10 to provide warnings to the user if a drug(s) being prepared and labeled is not suitable, or is not apparently suitable to be administered to the patient based on the patient information available. Based on the patient information, information in the formulary, the procedure identified by the user, or any combination thereof, other analyses can be performed, such as verification that the formulary or type of procedure selected as described above or gleaned from the content of a formulary tailored for a particular patient/surgical procedure is appropriate for this patient, patient drug allergies, drug to drug interactions, age related medication restrictions, etc. While performing such an analysis on the computer terminal 10 is one option, a more sophisticated analysis may be possible by communicating with a server included as part of the network 40 that receives individual requests for drug verification along with an indication for selecting the appropriate patient information from the computer terminal 10 and transmits a response to the computer terminal 10 that approves the use of the drug or provides the user with an appropriate warning that is displayed on touch-screen display 14.

Patient information associated with a procedure on computer terminal 10 as described above, can be used to provide drug tracking information for billing and patient records. As drugs are being prepared on computer terminal 10, the drug related information can be transmitted along with information required to associate the drug information with a patient to the EMR gateway server 47. The EMR gateway server 47 then transmits the drug related information along with associated patient information to the EMR 47 at the facility over network 40 using HL7 or another healthcare specific network protocol compatible with the EMR 47.

In another embodiment, the Patient information associated with a procedure on computer terminal 10 as described above, can be used to transmit information to a LIS (Laboratory Information System) 97 in the facility, shown in FIG. 3. The LIS 97 includes a network connected storage device such as a database server for example, that stores records of laboratory samples that are to be, or have been, subjected to medical testing at the laboratory in a computer-readable medium. In many surgical procedures, it is common to have a specimen removed from the patient that is sent to the laboratory for analysis. The specimen is often labeled by hand with patient information, tissue information, site information, date and time of extraction, attending physician, etc., and sent to the laboratory. The computer terminal 10 can allow the user to print a label with the same information that would normally be written by hand and transmit an electronic record of the data to the LIS 97 for storage, so the information on the label of the specimen will exactly match the information stored by the LIS 97 when the specimen arrives in the laboratory. The label produced by the computer terminal 10 can also include a machine-readable code on the label, such as a barcode for example, to allow a user to scan the barcode on the label upon receiving the sample at the laboratory and create an indication in a record stored by the LIS corresponding to that sample that the sample has been received by the laboratory for testing. Additional data such as the identification of the person who received the sample at the lab, the date/time of receipt, etc. . . . .

The computer terminals 10 can transmit data over the network to one or more of the terminals 42, 44 running the AT, a network-connected server, or other network resource, for example, that can be used to generate and analyze drug usage patterns based on procedure type, user or other relevant parameters. As drugs are being prepared by the user using a computer terminal 10, information about the drug including the drug name, concentration, container ID, date, time, user and procedure information can be stored in memory 24 on computer terminal 10 and then transmitted to the terminal(s) running the AT or a dedicated server. The information can then be post processed to extract the required information for determining usage patterns of drugs.

AIMS (Anesthesia Information Management Systems), also known as ARKS (Anesthesia Record Keeping Systems), include a server, represented generally at 77 in FIG. 3, that receives and stores drug usage information for each patient during a medical procedure to allow anesthesiologists to electronically record patient vital signs, drugs administered, important events that occurred during the surgery and other relevant information related to anesthesia administration and monitoring during a procedure. Many AIMS systems are programmed with a set of all drugs that could be administered in the operating room. This can be hundreds of drugs. When recording a drug in the AIMS 77, the user of the AIMS 77 usually navigates through multiple levels of menus to find the correct drug. A more efficient and accurate method of drug selection can be implemented using network 40 and computer terminal 10 to transfer drug information as they are prepared to the AIMS 77. The AIMS 77 user would then be presented with a short list of the drugs prepared on the computer terminal 10 when they record drug information on the AIMS 77. If the drug is not found on the short list of drugs prepared on computer terminal 10, then the user would have the option to access the full list of drugs stored on the AIMS 77. Although the email server 46, EMR 47, AIMS 77 and LIS 97 appear in FIG. 3 as separate, distributed computational platforms, it is to be understood that one or more of such platforms can be combined and embodied on a single computational platform without departing from the scope of the present invention.

Computer terminal 10 can optionally include a speaker 17 that plays audio files in response to the scanning of a barcode on a drug container by the scanner 18 during preparation of a label. Computer terminal 10 stores audio files or files that can be used to create audible sounds in memory 24. These audio files are executed by the computer terminal 10 to “speak” a drug name and concentration from the speaker 17 when a user scans a drug container using scanner 18. This provides audible confirmation to the user of the drug that was scanned. Other devices on the network that want to provide audible output of drug names, concentrations values and concentration units can transmit a message to computer terminal 10 over the network using a defined interface and message format to instruct computer terminal 10 to audibly “speak” the specified drug name and concentration information. The message can optionally include volume information. Alternately, the other device can transmit a message to computer terminal 10 using a defined interface and message format to select and receive the sound files from the computer terminal 10 and play the sound files locally on the device.

In another embodiment, the computer terminal 10 can transmit a list of prepared drug information over the network 40 to an administration terminal that is mounted near the point of drug administration to the patient. The administration terminal (not shown) can include a scanner similar to scanner 18 provided to the computer terminal 10, a display device for displaying the results of scanning a barcode or other machine-readable code, a processing component for converting a scanned code to the identity of the content of the container labeled with the barcode, and a network adaptor to receive the list of prepared drug information over the network. Optionally, the administration terminal can also include a speaker to audibly output the information pertaining to the content of the container labeled with the barcode when the barcode is scanned. The display device and/or the speaker can also optionally output a warning about the container and/or the drug therein in response to reading the barcode and determining that a warning is warranted.

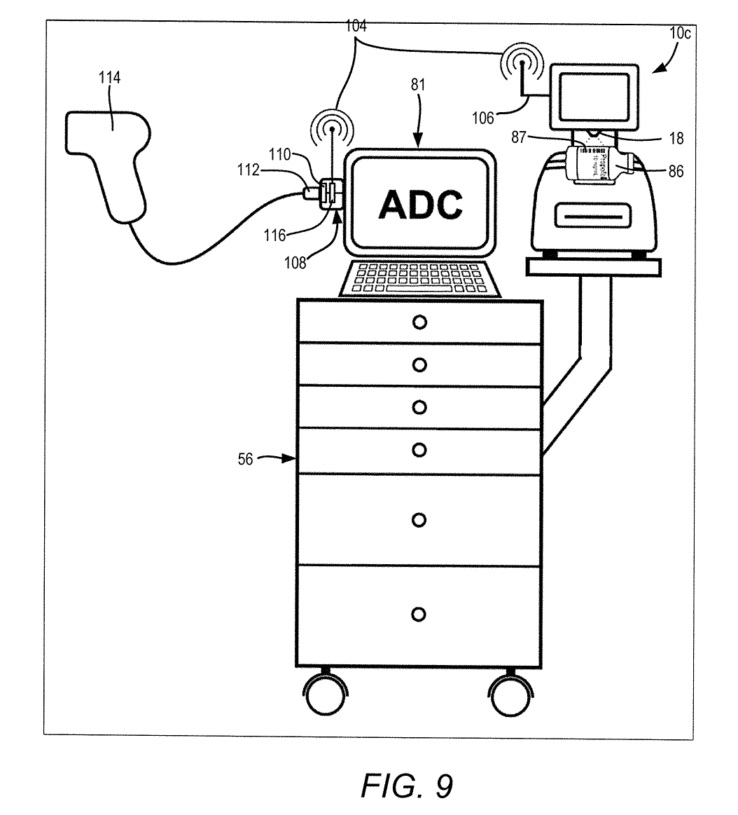

According to alternate embodiments such as that shown in FIG. 9, communications between the computer terminal 10c and the drug cart controller 81 can be established over a wireless communication channel 104. To establish the wireless communication channel 104, the computer terminal 10c can be equipped with a first interface 106 of a remote interface module (“RIM”) and the drug cart controller 81 can be equipped with a second interface 108 of the RIM that is compatible to communicate with the first interface 106. To facilitate retrofitting the existing computer terminal 10c and/or the drug cart controller 81 that lacks a built-in ability to communicate with the other with its respective interface, the first and/or second interface(s) 106, 108 can be external devices plugged into an appropriate port communication port. For example, the first interface 106 can optionally include a male USB connector to be installed in a female USB port provided to the computer terminal 10c. Likewise, the second interface 108 can optionally include a male USB connector to be installed in a female USB port provided to the drug cart controller 81. As an external device, the interface(s) 106, 108 can include their own housings, separate from the computer terminal 10c and/or the drug cart controller 81, respectively. Plugging any of the interfaces discussed herein into a USB port can optionally be powered via the USB port. According to alternate embodiments, the first and/or second interface(s) 106, 108 can optionally be integrated into the computer terminal 10c and/or drug cart controller 81, respectively, as an internal component.

Regardless of whether the first and second interfaces 106, 108 are externally or internally installed, the wireless communication channel 104 established between the interfaces 106, 108 can be compliant with a short-range local communication protocol such as Bluetooth, for example. Bluetooth is a wireless technology standard for exchanging data over short distances (e.g., less than 100 ft.) between devices by encoding the data using short-wavelength UHF radio waves in the ISM band from 2.4 to 2.485 GHz. However, the wireless communication channel 104 can be implemented according to any other desired wireless communication protocol, such as any of the 802.11 standards established by the IEEE, or any other suitable wireless technology such as optical communications that transmit and receive data using infrared light, for example.

The computer terminal 10c, the first interface 106 and the second interface 108 can each be assigned a unique identifier that uniquely identifies those devices (and distinguishes those devices from others) in the network 40 (FIG. 3). The computer terminal 10c can be configured to automatically recognize the first interface 106 in response to the first interface 106 being plugged into, or otherwise installed as part of the computer terminal 10c, or the first interface 106 can be manually selected by a user of the computer terminal 10c. The second interface 108 can be paired with the first interface 106 as a factory setting (e.g., a matched set) or a setting configured by a technician installing the equipment, or can be manually paired with the first interface 106 upon being selected by the user once both the first interface 106 and the second interface 108 have been installed on their respective device. Alternately, the pairing of second interface 108 with first interface 106 can be accomplished by connecting second interface 108 temporarily to computer terminal 10c using a different interface such as USB, for example, and allowing the user to manually select the second interface 108 from a list of one or more connected second interface 108 RIM devices that initiates the pairing between the second interface 108 and the first interface 106 on terminal 10c. In addition to establishing the wireless communication channel 104, the unique identifier assigned to the interfaces 106, 108 and the computer terminal 10c can be associated with data received over the wireless communication channel 104. The unique identifier of the transmitting device can be transmitted with, before and/or after the data is transmitted to identify that transmitting device as the source of the data being transmitted. According to alternate embodiments, the unique identifier of the device known to be involved in a communication over the wireless communication channel 104 can be logged or otherwise associated with the transmitted data by the device initiating such a communication.

As shown in FIG. 9, the second interface 108 also includes a port 110 that receives a connector 112 provided to a barcode scanner 114 or other peripheral of the drug cart controller 81. The connector 112 of the barcode scanner 114, while connected to the port 110, conveys signals from the barcode scanner 114 to the drug cart controller 81 via the second interface 108. A multiplexing circuit 116, which is similar to, or the same in structure and function as the multiplexing circuit 98 described above, conveys data received over the wireless communication channel 104 and from the barcode scanner 114 to the drug cart controller 81. The multiplexing circuit 116 can optionally translate or otherwise modulate data received over the wireless communication channel 104 to emulate data transmitted by the barcode scanner 114, thereby emulating a transmission from the barcode scanner 114. According to alternate embodiments, the computer terminal 10c can optionally be configured to transmit data that is compliant with the drug cart controller 81, thereby emulating a transmission from the barcode scanner 114. Emulating the barcode scanner 114 with the multiplexing circuit 116 or the computer terminal 10c allows a drug cart controller 81 that was not originally configured to receive and interpret data from devices other than the barcode scanner 114 to communicate with more than just the barcode scanner 114 via the second interface 108 as described in detail below. In other words, the second interface 108 allows a drug cart controller 81 originally configured to receive and interpret data only from the barcode scanner 114 to receive and interpret data from another source such as the computer terminal 10c. And since the second interface 108 also communicates with the barcode scanner 114, even drug cart controllers 81 with limited expansion or other communication ports can maintain communications with the barcode scanner 114 as well as communicating with the computer terminal 10c. Thus, if the information to be acquired by the barcode scanner 114 is available to the computer terminal 10c, scanning a barcode using the barcode scanner 114 can possibly be avoided as a repetitive procedure.

In alternate embodiments of the invention, the computer terminal 10c can optionally be configured to transmit data where a portion of the data is compliant with the drug cart controller 81 and the processing component 91 described above can be configured to identify and direct the compliant portion of the data received from computer terminal 10c to the drug cart controller 81 using multiplexing circuit 98.

The peripheral device is described herein as a barcode scanner 114 for the sake of clarity and brevity, but the present disclosure is not so limited. Instead, the peripheral can be any data entry device that, when connected to the drug cart controller 81, is operable to communicate with the drug cart controller 81.

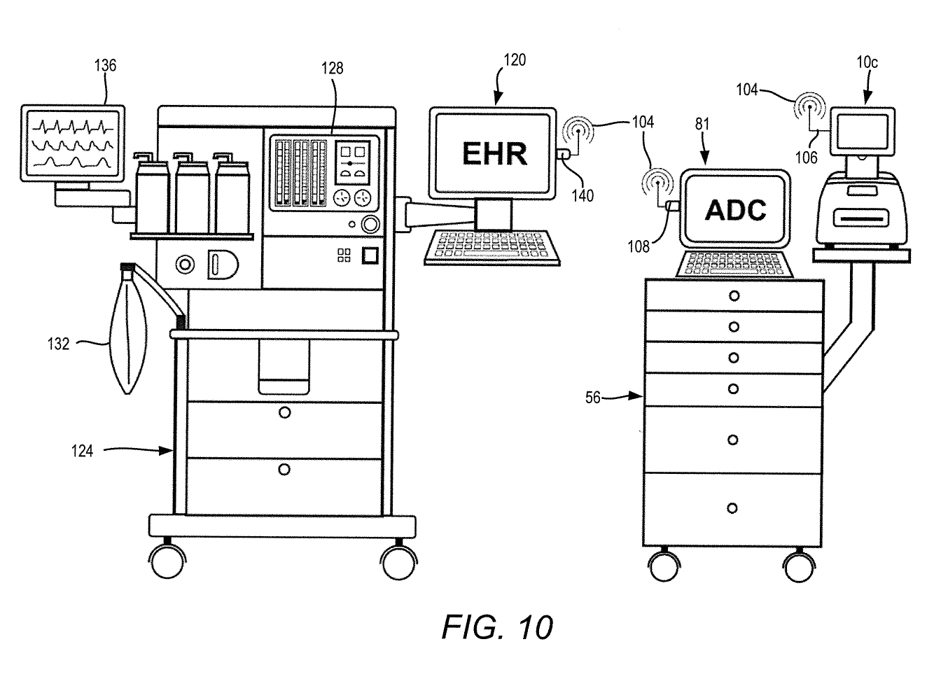

In addition to the drug cart 56, the computer terminal 10c can optionally be configured to communicate with a different device, or at least one more device in addition to the drug cart controller 81 via the RIM. This additional device is shown in FIG. 10 as an electronic health record (“EHR”) system 120. The EHR system 120 is a computer-implemented record system that maintains a database of patient records containing the details of medical care provided to those patients at a healthcare facility such as a hospital. As shown in FIG. 10, the EHR system 120 forms part of an anesthesia system 124 used in the administration of anesthesia to a patient during a surgical or other medical procedure. As an example, the anesthesia system 124 can be a so-called continuous-flow anesthesia gas machine, which includes a gas regulator 128 that supplies an accurate and continuous supply of medical gases (e.g., oxygen, nitrous oxide, etc.), in a desire ratio with an anesthetic vapor (such as isoflurane, for example). The gas regulator delivers such gases to the patient at a suitable pressure and flow to maintain the patient in a sedated state during the medical procedure. A ventilator 132 can be provided to perform artificial respiration for the patient, while a patient monitoring system 136 receives signals from sensors applied to the patient to monitor vital signs and other parameters indicative of the patient's condition during a medical procedure. Examples of the sensors and vital signs monitored can include at least one, a plurality, and optionally all of: a blood pressure cuff to monitor blood pressure, electrocardiogram leads to monitor heart rate, a pulse oximeter to measure blood oxygen levels, a thermometer to sense the patient's temperature, and an oxygen and/or carbon dioxide analyzer to measure the amount of oxygen and/or carbon dioxide, respectively, inhaled and exhaled by the patient.

The continuous-flow gas machine type of anesthesia system 124 is offered above merely as an example. Other types of anesthesia systems 124, possibly including a patient monitoring system 136 that monitors one or more vital signs and/or other parameters different from those above also fall within the scope of the present disclosure. Additionally, the EHR system 120 can optionally be configured to include a stand-alone terminal that may communicate with the anesthesia system 124, or may lack some or all of the ability to communicate with the anesthesia system 124. EHR systems 120 lacking such a communication ability would require separate (and possibly manual) entry of data concerning the administration of drugs or other patient care provided during the medical procedure, if not for the communications, including those over the communication channel 104, described herein.

Similar to the RIM discussed above, the RIM utilized in the embodiment illustrated in FIG. 10 includes an external adaptor or third interface 140 installed in a USB or other communication port of the EHR system 120. The third interface 140 is also compatible with the other components of the RIM to communicate via a short range communication protocol such as Bluetooth, for example, via any of the 802.11 standards established by the IEEE, or any other suitable wireless technology.

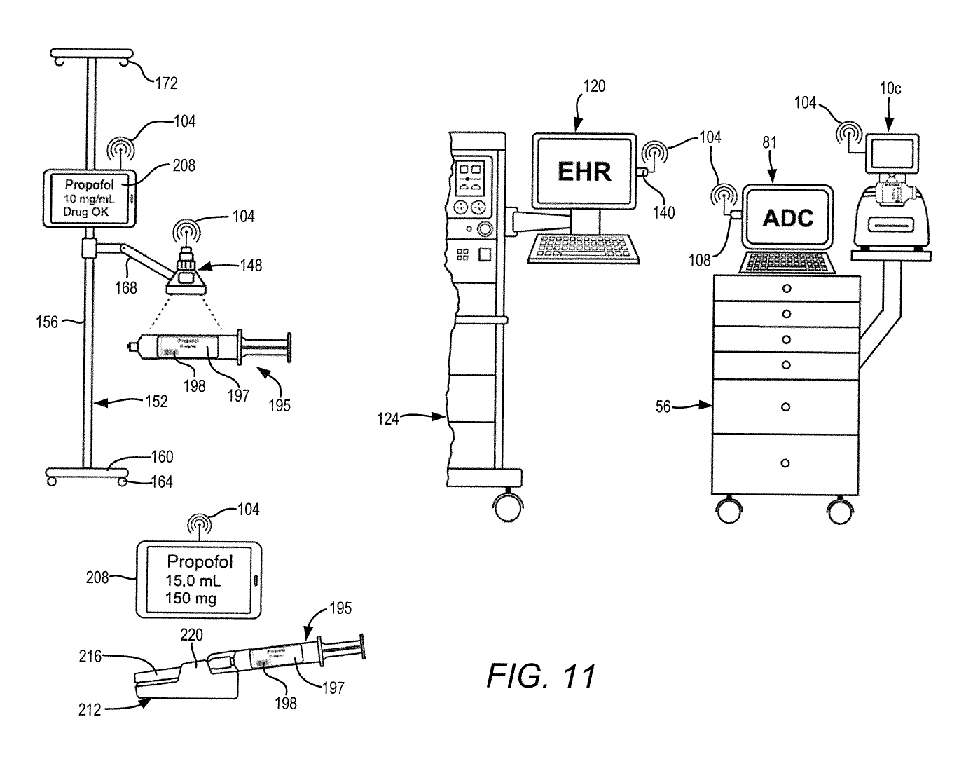

At locations such as an operating room, in-patient room, or other patient-care location, a barcode 198 (FIG. 11) on a label 197 provided to a syringe 195 or other delivery container such as an IV bag, for example, is to be interrogated using a compatible barcode reader to allow for computer identification of the drug being administered as described in detail below. Although other computer-readable codes such as those stored by RFID tags, for example, can be used instead of a barcode, use of a one-dimensional barcode is described for the illustrative embodiments for the sake of clarity and brevity. With reference to FIG. 11, to facilitate drug recognition and optionally documentation at a point of care where the drug is to be administered, a remote barcode reader 148 can be supported by a portable stand 152. The stand 152 includes an upright pole 156 that can optionally have an adjustable height and/or include a bracket 168 that can be adjusted to support the remote barcode reader 148 at a desired height for use. A base 160 with a plurality of casters 164 allows the stand 152 to be rolled to the point of care. One, or a plurality of IV hooks 172 can optionally be provided adjacent to an upper region of the stand 152 to hang an IV bag containing a drug or other substance to be administered intravenously over a prolonged period. Such IV bags (not shown) can also be a drug delivery container provided with a label 197 bearing a barcode 198 as described below using the syringe 195 as the example. In alternate embodiments, the remote barcode reader 148 can be mounted to the mounting hardware used to attach the EHR system 120 to the anesthesia system 124 or directly to the anesthesia system 124. Additionally, the remote barcode reader 148 can contain a mounting base designed for placement on a flat surface such as a table, tray or ledge for example, that is a part of the anesthesia system 124.

A drug infusion pump (not shown) can optionally be supported by the stand 152 to control administration of a drug at a controlled rate to achieve a predefined dose and/or volume over a prescribed period of time.

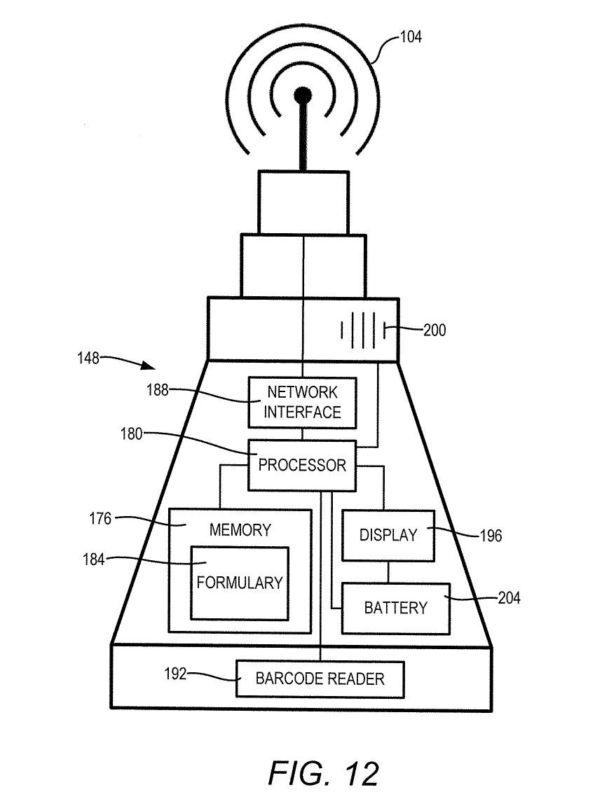

A partially cutaway schematic view of the remote barcode scanner 148 is shown in FIG. 12. As shown, the remote barcode scanner 148 includes a non-transitory computer readable memory 176 that stores logic executable by a processor 180 to perform the functions of the remote barcode scanner 148 described herein. Additionally, the memory 176 can optionally store a drug formulary 184 that can be queried by the processor 180 locally, without communicating with a remote device such as terminal 10c, for example, via the network interface 188, to obtain information pertaining to a drug identified by a barcode scanned using a barcode reader 192. According to alternate embodiments, the memory 176 can optionally lack a local drug formulary, requiring the processor 180 to initiate the retrieval of information pertaining to a drug scanned using the barcode reader 192 from a remotely-stored formulary via the network interface 188. In such case, the information retrieved can be stored locally in memory 176 for future use or can be utilized immediately and handled as transient data when no long-term local storage is required as is typical of streaming data such as digital sound, for example. A display 196 can be provided to visually convey information pertaining to the drug, while a speaker 200 can audibly announce such information. The embodiment of the remote barcode scanner 148 in FIG. 11 is powered by a battery 204, but other embodiments of the remote barcode scanner 148 can operate on power supplied through a powered communication port (e.g., USB port) of a locally-connected terminal or other USB power source, or on power supplied by a wall outlet connected to a public electric utility.

The processor 180 can be any computational unit such as an ASIC, programmable logic controller, digital signal processor, or any other suitable control circuit capable of executing the logic stored by the memory 176. The memory 176 can be any non-transitory, computer-readable medium such as a hard disk drive, read-only memory (“ROM”), random access memory (“RAM”), or any other suitable data storage device, or any combination thereof.

The barcode reader 192 can optionally be any non-contact device that can interrogate and read a computer-readable code applied to a drug container such as a barcode, RFID tag, and the like. For the sake of brevity, the computer readable code is shown in the drawings and described from this point forward as a one-dimensional (“1D”) barcode. The barcode reader 192 can optionally be configured with optics or image processing algorithms specifically designed to read a barcode applied to, and wrapped at least partially around an arcuate surface such as the barrel of a syringe 195.

The display 196 can include a low-power display screen such as a liquid crystal display (“LCD”), organic light emitting diode (“OLED”) display, or other display screen that can display graphic and/or textual information. According to alternate embodiments, the display 196 can include a variable-color light source such as a multi-color LED that emits visible light at wavelengths representing a plurality of different colors, each conveying different feedback concerning the drug to a user visually. Yet other embodiments of the display 196 can include a plurality of discrete light sources, such as LEDs, each emitting a different color of light. Again, the different colors can provide different visual indications pertaining to use of the drug, the status of the remote barcode reader 148 (e.g., connection over the wireless communication channel 104 has been lost, drug not found in the local formulary 184, etc.), or any other condition that could affect use of the remote barcode reader 148 and/or administration of the drug.

The network interface 188 can be integrated as part of the structure of the remote barcode scanner 148, but can also be an add-on module similar to the interfaces 106, 108, 140 discussed above. For example, the network interface 188 can establish communications between the processor 180 and one or more of the interface(s) 106, 108, 140 over a wireless communication channel 104 using a short-range local communication protocol such as Bluetooth, for example, or any other desired wireless communication protocol, such as any of the 802.11 standards established by the IEEE, or any other suitable wireless technology.

Referring again to FIG. 11, in addition to the remote barcode scanner 148, the stand 152 can optionally support a display device 208 such as a notebook computer, computer monitor, tablet computer, etc. The display device 208 that includes its own interface to communicate with at least the remote barcode scanner 148 via the wireless communication channel 104. Although the wireless communication channel 104 utilized by each device herein is identified using the same reference numeral, the wireless communication channel 104 utilized by each device can be independently selected for communications with the specific counterpart. For example, the remote barcode scanner 148 can communicate with the display device 208 using Bluetooth, and communicate with the computer terminal 10c using IEEE 802.11ac. However, using a common wireless communication channel 104 affords improved communications between many devices.

The environment appearing in FIG. 11 also includes a bolus injection monitor 212 that senses a quantity of a drug injected, as a single dose or as a plurality of doses occasionally administered, during a medical procedure. The monitor 212 includes a sensor component 216 that can sense the flow of the drug from the syringe 195 using a capacitive, optical, ultrasound or other type of signal indicative of the rate and/or quantity of fluid flowing through the needle of the syringe 195. A barcode reader 220 is also provided to the monitor 212 at a position to read the 1D barcode 198 while the syringe 195 is being introduced to the monitor 212, or once the syringe 195 is installed on the monitor 212. The barcode reader 220 can also be configured to read a 2D barcode on label 197 or a secondary label (not shown) affixed to the syringe. The monitor 212 communicates via the wireless communication channel 104 with the display device 208 which, can be a notebook computer, computer monitor, tablet computer, etc., and optionally the same display device 208 supported by the stand 152, to convey information indicative of the drug identity and optionally additional information including, but not limited to, at least one of: total volume in the syringe 195, total volume administered as measured by the sensor 216, total dose in the syringe 195, total dose administered as measured by the sensor 216, drug concentration, etc.

The bolus injection monitor 212 (FIG. 11) can optionally be configured with an attached or integrated display 225 that can include a low-power display screen such as a liquid crystal display (“LCD”), organic light emitting diode (“OLED”) display, or other display screen that can display graphic and/or textual information. According to alternate embodiments, the display 225 can include a variable-color light source such as a multi-color LED that emits visible light at wavelengths representing a plurality of different colors, each conveying different feedback concerning the drug to a user visually. Yet other embodiments of the display 225 can include a plurality of discrete light sources, such as LEDs, each emitting a different color of light. Again, the different colors can provide different visual indications pertaining to the type of drug, use of the drug, the status of the remote barcode reader 148 (e.g., connection over the wireless communication channel 104 has been lost, drug not found in the local formulary 184, etc.), or any other conditions that could affect the operation of the bolus injection monitor 212. Alternately, the aforementioned display functions can be performed by a separate display device 208 such as a notebook computer, computer monitor, tablet computer, etc. that is connected wirelessly, or via a cable, to the bolus injection monitor 212 and is integral to the operation of the bolus injection monitor 212.

In use to prepare a label for a syringe 195, the barcode 87 appearing on the label of a drug vial 86 is scanned using the barcode scanner 18 provided to the computer terminal 10c as shown in FIG. 9. As a result of scanning the barcode 87, the computer processor 22 (FIG. 2) initiates a query of formulary 36 in an attempt to identify the drug in the drug vial 86. For example, the barcode 87 can optionally encode the NDC of the drug vial 86 or other uniquely-identifying number, and the query of the formulary 36 can be performed based on the NDC. The information required to print the syringe label 197 (e.g., FIG. 11) can be retrieved from the formulary 36 and the label 197 printed by the printer 26 (FIG. 2).

Certain information obtained by the computer terminal 10c as a result of scanning the barcode 87 on the drug vial 86 (e.g., the NDC, other information retrieved from the formulary 36, information retrieved from a remote source based on the NDC and/or information retrieved from the formulary, etc.) can also be utilized by another device in communication with the computer terminal 10c, optionally for a purpose other than providing healthcare services to the patient. Such information is referred to hereinafter as “Vial Information” as it was obtained by the computer terminal 10c as a result of scanning the barcode 87 on the drug vial 86. To be clear, Vial Information does not include only the information encoded by the barcode 87, but also includes, and is not limited to: any information retrieved from the formulary 36 based on the scan of the barcode 87, and any information obtained from any other source as a result of scanning the barcode 87 on the drug vial 86 during the process of printing the syringe label 195. For example, the computer terminal 10c can transmit at least a portion of the Vial Information over the wireless communication channel 104 to be received by the interface 108 provided to the drug cart controller 81 for purposes of documenting the consumption of drugs from the drug cart 56.

The computer terminal 10c prepares a communication to be transmitted over the wireless communication channel 104 via the interface 106. Included in the communication is at least a portion, optionally less than the entirety of the Vial Information, or optionally all of the Vial Information. The portion of the Vial Information can optionally be combined with additional information, other than Vial Information (e.g., information input by a user into the computer terminal 10c indicating a number of vials corresponding to the scanned barcode 87 that were removed from the drug cart 56, or information related to the patient that was entered into or received by computer terminal 10c, or information received from other devices or systems connected to the same network as terminal 10c in the healthcare institution, etc.) to be transmitted to the intended recipient which, in this example, is the drug cart controller 81.

The computer terminal 10c can optionally be configured with a rules package defining how the processing component 22 (FIG. 2) of the computer terminal 10c prepares the communication. For example, the computer terminal 10c can be configured specifically to communicate with the drug cart controller 81. According to such an embodiment, the computer terminal 10c can format the portion of the Vial Information and any additional information into a format that the drug cart controller 81 would expect to receive from the hand-held scanner 114, for example. This format utilized by the computer terminal 10c can optionally be specific to the drug cart controller 81, and not used by another device with which the computer terminal 10c can communicate. The computer terminal 10c transmits the data over the wireless communication channel 104 to be received by the interface 108 provided to the drug cart controller 81.

Upon being received by the interface 108, the received data is conveyed to the drug cart controller 81. The drug cart controller 81 can utilize this received data to adjust a quantity of the drug corresponding to the scanned barcode 87 remaining in the drug cart 56. If a total of two vials 86 were removed, the inventory maintained by the drug cart controller 81 can be decremented by two.

As another example, the computer terminal 10c can be configured specifically to transmit at least a portion, optionally all or less than an entirety of the Vial Information over the wireless communication channel 104 to be received by the interface 140 provided to the EHR system 120. Just as in the previous example, the computer terminal 10c can prepare and format the data to be transmitted to the EHR system 120 to update the patient record maintained by the EHR system 120. Drug administration information including the name of the drug being to be administered, the date of administration and other such information transmitted by the computer terminal 10c to be tracked for documenting the medical procedure performed on the patient can be added to the patient's medical record. In addition to recording information related to a patient's healthcare, invoices accurately reflecting the drugs consumed can be prepared based on this information. The configuration of the computer terminal 10c to transmit data to the EHR system 120 via the wireless communication channel 104 can be specific to the format required by the EHR system 120.

Once the label 197 for the syringe 195 has been prepared to include the 1D barcode 198, this barcode 198 can subsequently be scanned as part of the provision of healthcare to the patient. The barcode 198 generated by the computer terminal 10c can be scanned by the barcode reader 192 (FIG. 12) of the remote barcode reader 148 at, or near where the drug is to be administered to the patient. For example, the stand 152 can be transported into an operating room to allow clinicians to scan the barcode 198 with the remote barcode reader 148 in close proximity to the patient. For the embodiment of the remote barcode reader 148 shown in FIG. 12, which includes a locally-stored formulary 184, the processor 180 can be configured to initiate a drug lookup from the formulary 184 in response to the barcode 198 applied to the syringe 195 being scanned in an attempt to identify the drug. The entry identified in the formulary as being the most-likely match can be retrieved and a sound file locally stored for that entry in the memory 176 can be played via the speaker 200 to alert the clinician to the determined identity of the drug, a concentration of the drug in the prepared syringe 195, an expiration date of the drug in the syringe 195, a warning relating to the administration of the drug to the patient, or any other condition related to the drug in the syringe 195, optionally as administered to the specific patient on whom the medical procedure is being performed.

The remote barcode reader 148 can optionally, in addition to or in lieu, of the audible announcement, generate a visible notification to the clinician through operation of the display 196. The information displayed can include any of the information that can be audibly announced by the speaker 200.

The remote barcode reader 148 can be configured to communicate with the computer terminal 10c subsequent to, and optionally in response to, reading the barcode 198 used to label the syringe 195. For example, the remote barcode reader 148 can transmit at least a portion of the information derived from scanning the barcode 198 to the computer terminal 10c. The remote barcode reader 148 can transmit an identification number such as the NDC, a unique internal identifier that is a proprietary, internal identifier of the healthcare facility that uniquely identifies the syringe 195, or any other drug identifying information to the computer terminal 10c.

Such information can be transmitted by the remote barcode reader 148 to obtain confirmation information concerning the drug in a return transmission. The computer terminal 10c can be configured to compare the information transmitted by the remote barcode reader 148 to data contained in the formulary 36 (FIG. 2) or other database to determine whether the drug is correctly labeled with the barcode 198, has not expired, or otherwise confirm any other property concerning the drug in the syringe 195. The result of the comparison performed by the computer terminal 10c can then be transmitted back to the remote barcode reader 148 so the confirmation can be passed onto the clinician, and data associated with administration of the drug can be transmitted by the remote barcode reader 148 to the EHR system 120 or other intended recipients, such as other devices in communication with the remote barcode reader 148 over wireless communication channel 104 or over a USB channel for example, as a result.

According to alternate embodiments, the confirmation comparison can be performed by the remote barcode reader 148, or any other device accessible via the wireless communication channel 104, instead of the computer terminal 10c. According to such embodiments, the remote barcode reader 148 can transmit the information required to elicit the responsive transmission by the computer terminal 10c or other device including the information required to confirm the information pertaining to the drug determined by scanning the barcode 198 on the syringe 195.

Regardless of the device that performs the comparison, data received by the remote barcode reader 148 as a result of reading the barcode 198 and communicating with a remote terminal via the wireless communication channel 104 can be used by the remote barcode reader 148 to supplement, update or otherwise alter the information stored locally in the memory 176. For example, if a drug entry could not be located in the local formulary 184 stored by the remote barcode reader 148 and the response from the computer terminal 10c includes drug information of that missing entry, the formulary 184 can be updated to add the drug in question.

The above use case involved an embodiment of the remote barcode reader 148 that includes a locally-stored formulary 184 as part of the remote barcode reader 148 itself. In other words, the formulary 184 is accessible by the processor 180 even in the absence of any network connection that would enable the remote barcode reader 148 to communicate with another device. However, according to alternate embodiments of the remote barcode reader 148, the memory 176 can optionally lack a full complete drug formulary 184 that includes the sound files or displayable content. To use such embodiments, the network interface 188 can transmit a request to the computer terminal 10c via the wireless communication channel 104 in response to scanning the barcode 198 provided to the label 197 on the syringe 195. Such a communication conveys to the computer terminal 10c sufficient information to enable the computer terminal 10c to identify the drug in question from the formulary 36 stored in the memory 24 (FIG. 2), from another database stored by the memory 24, or from another source accessible to the computer terminal 10c. The computer terminal 10c can then respond to the remote barcode reader 148 via the wireless communication channel 104 with information such as the drug's identity, an expiration date and/or time of the drug in the syringe 195, an audio clip to be played by the speaker 200 (FIG. 12), content to be displayed by the display 196, instructional information detailing any special or uncommon instructions for administration of the drug, any other information, or any combination thereof.

The remote barcode reader 148 can be configured specifically to communicate with the various devices that can be reached via the wireless communication channel 104. Just as the computer terminal 10c formats data to be transmitted to the drug cart controller 81, for example, the remote barcode reader 148 can tailor the data to be transmitted to an intended recipient specifically for that intended recipient. This includes not only the format, but the information that is to be transmitted.

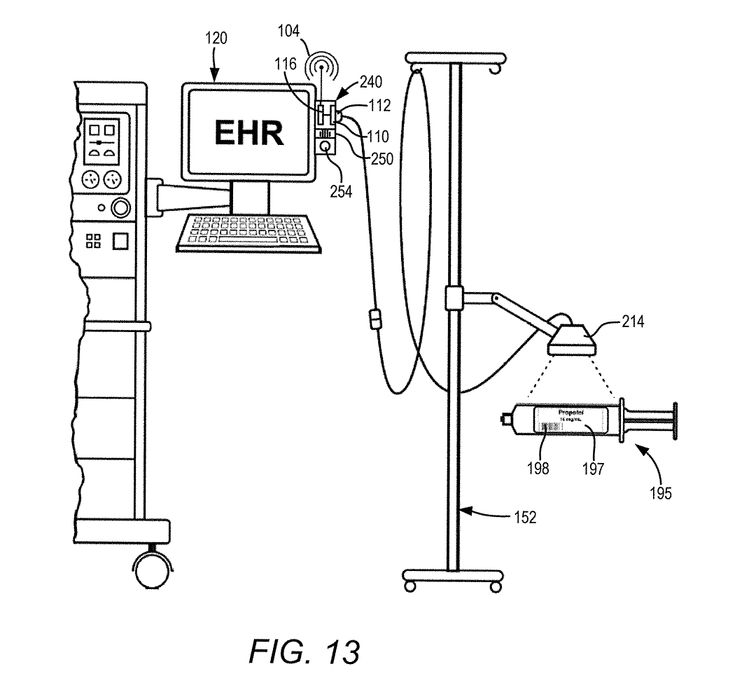

An alternate embodiment of the third interface 140 provided to the EHR system 120 is shown in FIG. 13. According to the present embodiment, the interface 240 includes a pole-mounted barcode scanner 214 installed in a USB or other communication port of the EHR system 120. The stand 152 described above, or a similar support structure, can optionally be used to support the barcode scanner 214. The interface 240 is also compatible with the other components of the RIM (e.g., interfaces 106, 108) to communicate over the wireless communication channel 104 via a short range communication protocol such as Bluetooth, for example, via any of the 802.11 standards established by the IEEE, or any other suitable wireless technology. The interface 240 also includes a port 110 that receives a connector 112 provided to a barcode scanner 214 or other peripheral of the EHR system 120. The barcode scanner 214 can be a simple scanner such as the handheld scanners 84, 114 described above, that lacks the speaker, memory storing a formulary, etc. provided to the remote barcode reader 148. The connector 112 (e.g., male USB plug) of the barcode scanner 214, while connected to the port 110 (e.g., female USB port), conveys signals from the barcode scanner 214 to the EHR system 120 via the interface 240. A multiplexing circuit 116, which is similar to, or the same in structure and function as the multiplexing circuit 98 described above, conveys data received over the wireless communication channel 104 and from the barcode scanner 214 to the EHR system 120. The multiplexing circuit 116 can optionally translate or otherwise modulate data received over the wireless communication channel 104 to emulate data transmitted by the barcode scanner 214, thereby emulating a transmission from the barcode scanner 214.

In another embodiment of the invention, the interface 240 can be mounted on or near the barcode scanner 214 instead of on or near EHR system 120. For example, a USB cable can be connected to EHR system 120 that extends to a USB port on interface 240. An optional short USB cable can connect barcode scanner 214 to a second USB port on interface 240. Alternately, a direct connection between the second USB port on interface 240 and the barcode scanner 214 can be made instead of using a USB cable. The placement of interface 240 in close proximity to barcode scanner 214 can be more convenient for users by providing visual and audio feedback of the drug being scanned at a point that is physically closer to where barcode 198 on syringe 195 is scanned on barcode scanner 214 by the user.

The interface 240, as an externally installed peripheral to the EHR system 120, includes at least one of a speaker 250 and a light source 254 to provide a visible indication of the current state of the drug in the syringe 195. The light source 254 can include a variable-color light such as a multi-color LED that emits visible light at a plurality of different wavelengths representing different colors, each conveying different feedback concerning the drug. Yet other embodiments of the light source 254 can include a plurality of discrete light sources, such as individual LED bulbs or clusters, each emitting a different color of light. Again, the different colors can provide different visual indications pertaining to the usability of the drug 148 (e.g., whether the drug could be identified, whether the drug in the syringe 195 has expired, etc.), or any other condition that could affect administration of the drug.

In use, the barcode 198 on the label 197 provided to the syringe 195 is scanned using the barcode scanner 214 supported by the stand 152, and the barcode scanner 214 transmits a signal indicative of the scan to the interface 240 through the USB port 110. The signal, upon being received by the interface 240, can be temporarily stored by the interface 240 before being conveyed to the EHR system 120 until the interface determines that the drug in the syringe 195 is suitable to be administered to the patient. To determine this, the interface 240 transmits a signal indicative of the drug over the wireless communication channel 104 to be received by the interface 106 of the computer terminal 10c. This transmission itself can serve as a request for confirmation that the drug in the syringe 195 has not expired and is otherwise suitable to be administered. According to alternate embodiments, the transmission can optionally include a specific request for certain information required to be received by the interface 240 before the interface 240 can indicate that administration of the drug using the syringe 195 can proceed.

Using at least a portion of the information include in the transmission, the computer terminal 10c determines whether all conditions for use of the drug (e.g., the drug in the syringe 195 has not expired, etc.) known to the computer terminal 10c have been satisfied. The computer terminal 10c then transmits a response via the interface 106 over the wireless communication channel 104 to be received by the interface 240. If, based on the response received from the computer terminal 10c, the drug is suitable to be administered to the patient, the speaker 250 can optionally announce the name of the drug audibly, possibly with other information such as the drug concentration, for example. The light source 254 can optionally emit light of a certain color to indicate that the drug is suitable to be administered to the patient. Once the transmission from the computer terminal 10c including information indicating that administration of the drug can continue, the interface 240 can then convey the drug information obtained as a result of scanning the barcode 198 on the label 197 for the drug to the EHR system 120.

If, based on the response received from the computer terminal 10c, the drug is suitable to be administered to the patient however, the speaker 250 can optionally announce a warning, notifying the clinician of the possibility of a problem with the drug, and that the drug should not be administered from the syringe 195. The light source 254 can optionally emit light of another color to indicate that the drug is not suitable to be administered to the patient. For example, a specific syringe 195 should only be used on a single patient. Reuse of a syringe on different patients is a condition that can be identified by computer terminal 10c that triggers an alert to the user by displaying a yellow or red color on light source 254 and optionally producing an appropriate audible alert such as “Possible syringe re-use” on speaker 250.

In another embodiment, the remote barcode scanner 148 (FIG. 12) can be used to read barcodes printed on sources other than drug label 198 and perform specific actions based on rules stored in memory 176 and executed by processor 180. For example, the user can read the barcode on a patient wristband entering the operating room using barcode scanner 192. Rules stored in memory 176 can identify the barcode as being from patient wristband (not shown) by analysis of the barcode symbology or data encoded within then barcode. Rules logic can also define an analysis to parse the data encoded in the barcode to identify the specific ID associated with the patient. The patient ID can optionally be stored in memory 176 and also be transmitted via wireless interface 104 to computer terminal 10c where it can used to perform additional patient specific drug related processing such as recording drugs used a particular patient and detecting when the syringe 195 was previously used on other patient.

Additional embodiments include a user initiated trigger mechanism on the remote barcode scanner 148 (FIG. 12) that sends a signal via wireless interface 104 to a remote device such as computer terminal 10c or other remote devices accessible via wireless interface 104, that trigger a behavior, change configurable settings or perform a specific function on the remote device. For example, the user can activate a button (not shown) on remote barcode scanner 148 to send a signal indicative of the start of a new medical case involving a different patient to computer terminal 10c. Alternately, the user could scan a barcode encoded with specific data or using a specific symbology that is recognized by rules logic processed by remote barcode scanner 148 and initiates an event similar to activating a button.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A remote barcode reader that is to communicate with a terminal in a medical network, the remote barcode reader comprising:
an optical barcode scanner that transmits a signal indicative of a barcode in response to interrogating the barcode;
a non-transitory computer-readable memory that stores, at least temporarily, information obtained in response to reading the barcode;
a network interface that communicates wirelessly over a wireless communication channel with a remote device in a medical network to obtain information pertaining to a drug that is identifiable from the information obtained in response to reading the barcode; and
a processor that initiates the transmission of a communication based on the information obtained in response to reading the barcode to the remote device over the wireless communication channel via the network interface, and delays transmitting at least a portion of the information obtained in response to reading the barcode until at least a time when a response including information related to the drug is received from the remote device.

2. The remote barcode reader of claim 1, wherein the processor is configured to interfere with transmission of the at least a portion of the information obtained in response to reading the barcode to the terminal if the response indicates a problem associated with administration of the drug to a patient.

3. The remote barcode reader of claim 2 further comprising a warning system comprising an audible warning device, a visible warning device, or both the audible warning device and the visible warning device that alerts a clinician if the response indicates the problem associated with administration of the drug to the patient.
